(19) United States
(12) Patent Application Publication
(10) Pub. No.: US 2024/0009325 A1
(43) Pub. Date: Jan. 11, 2024

(54) METHODS AND FORMULATIONS FOR GENE THERAPY, AND FOR COMBINING GENE THERAPY WITH DITPA TREATMENT, OF ALLAN-HERNDON-DUDLEY SYNDROME

(71) Applicants: PriZm, LLC, Chandler, AZ (US); The University of Chicago, Chicago, IL (US); The University of Miami, Coral Gables, FL (US); Cedars-Sinai Medical Center, Los Angeles, CA (US)

(72) Inventors: Samuel REFETOFF, Chicago, IL (US); Roy WEISS, Miami Beach, FL (US); Khemraj HIRANI, Chandler, AZ (US); Clive SVENDSEN, Pacific Palisades, CA (US); Pablo AVALOS, West Hollywood, CA (US); Gad VATINE, Ramat Gan (IL)

(21) Appl. No.: 18/350,077

(22) Filed: Jul. 11, 2023

Related U.S. Application Data
(60) Provisional application No. 63/388,235, filed on Jul. 11, 2022.

(51) Int. Cl. A61K 48/00; A61K 31/192; A61P 25/00

(52) U.S. Cl. CPC A61K 48/005; A61K 31/192; A61K 48/0016; A61P 25/00

(57) ABSTRACT

The present disclosure is directed to methods of treating Allan-Herndon-Dudley syndrome comprising administering 3,5-diiodothyropropionic acid (DITPA) to a subject in need thereof, and to administering gene therapy to the subject by introducing normal human MCT8 into the subject's cells in order to increase T3 in the subject's brain.

### CROSS-REFERENCE TO RELATED APPLICATION

This application claims the benefit of U.S. Provisional Patent Application No. 63/388,235, filed on Jul. 11, 2022, the content of which is incorporated herein by reference in its 10 entirety.

### GOVERNMENT LICENSE RIGHTS

This invention was made with government support under Grant No. DK15070 awarded by the National Institutes of Health (NIH). The government has certain rights in the invention.

### TECHNICAL FIELD

The present disclosure is directed to methods of gene therapy for treating a subject with Allan-Herndon-Dudley syndrome, and to methods of combining administration of 3,5-diiodothyropropionic acid (DITPA) to a subject with Allan-Herndon-Dudley syndrome with gene therapy administered to the subject.

### BACKGROUND ART

Allan-Herndon-Dudley Syndrome (“ARDS”) is an X-linked recessive developmental disorder causing intellectual disability and movement issues in males. Specifically, patients with ARDS have a mutant SLC16A2 gene resulting in a malformed monocarboxylate transporter 8 (“MCT8”) protein. MCT8 is a thyroid hormone cell membrane transporter.

In 2004 it was demonstrated that a severe neuropsychomotor defect observed in males in 1944 by Allan Herndon and Dudley¹ was caused by defects of MCT8 gene (also known as SLC16A2)². These individuals, mostly children, cannot walk or talk because of deficiency in the active thyroid hormone T3 in brain. However, at the same time, these children have increased metabolism and failure to gain weight because the excess of T3 present in blood entered other body tissues though alternative transporters.

Symptoms of ARDS are caused by a lack of cellular uptake of the thyroid hormone triiodothyronine (“T3”), which is normally transported across the cell membrane by MCT8. This MCT8 deficiency leads to a lack of T3 in tissues that need T3 to function properly contributing to an accumulation of T3 in the blood serum. The other thyroid hormone thyroxine (“T4”) usually remains at normal serum levels in ARDS patients but may also be slightly reduced from a normal level. Thyroid stimulating hormone (“TSH”) is normal to slightly elevated in ARDS patients.

The thyroid hormone analogue, diiodothyropropionic acid (DITPA), given to mice deficient in MCT8 had equal suppressive effect on thyroid stimulating hormone (TSH) as that observed in normal mice³. This suggested that DITPA can enter cells in the absence of MCT8. Administration of DITPA to 4 MCT8 deficient children, on compassionate basis, showed reduction and normalization of the high blood T3, thus ameliorating the hypermetabolism and nutrition but not the neuropsychomotor deficit⁴.

Introduction of the normal human MCT8 into cells by the means of the viral vector AAV9 to newborn Mct8 deficient mice was able to increase T3 in their brain and induce a T3-mediated effect⁵. However, these mice had no neurological deficit owing to an alternative transporter. Recently the same gene therapy was given to peripubertal Mct8 deficient-dKO mice that have neurocognitive abnormalities. It corrected the neurological abnormalities, learning and recall abilities but not the high blood T3 causing the increased metabolism⁶.

Currently, no treatment for ARDS has been approved by the United States Food and Drug Administration. Clinical trials have been completed for the drug, triiodothyroacetic acid (“TRIAC”), for use in the treatment of ARDS. However, TRIAC shares a close structural similarity to T3, which makes it difficult to accurately assess T3 serum levels. Further, TRIAC has been shown to significantly reduce T4 serum levels. See, Groeneweg et al. Lancet Diabetes Endocrinol. 2019 September; 7(9); 695-706.

A combined gene and DITPA treatment should correct both neuropsychomotor and metabolic defects that each treatment alone could not achieve, providing full rescue of the genetic defect.

### SUMMARY

Allan-Herndon-Dudley Syndrome (“ARDS”) is an X-linked recessive developmental disorder causing intellectual disability and movement issues in males. Specifically, patients with ARDS have a mutant SLC16A2 gene resulting in a malformed monocarboxylate transporter 8 (“MCT8”) protein. Symptoms of ARDS are caused by a lack of cellular uptake of the thyroid hormone triiodothyronine (“T3”), which is normally transported across the cell membrane by MCT8. This MCT8 deficiency leads to a lack of T3 in tissues that need T3 to function properly contributing to an accumulation of T3 in the blood serum. The other thyroid hormone thyroxine (“T4”) usually remains at normal serum levels in ARDS patients but may also be slightly reduced from a normal level. Thyroid stimulating hormone (“TSH”) is normal to slightly elevated in ARDS patients.

3,5-diiodothyropropionic acid (“DITPA”) is another thyroid hormone analog that has been studied for treatment of ARDS. However, as mentioned above, DITPA has not yet been approved for use in the treatment of ARDS. This lack of approval may be due to a lack of effective dosing regimens, stable and effective compositions, and extensive pharmacological assessments. While WO/2012/171065, published Dec. 20, 2012, attempts to establish DITPA dosing regimens for ARDS patients, this publication offers only theoretical examples.

Administration of DITPA to 4 MCT8 deficient children, on compassionate basis, showed reduction and normalization of the high blood T3, thus ameliorating the hypermetabolism and nutrition but not the neuropsychomotor deficit⁴.

Introduction of the normal human MCT8 into cells by the means of the viral vector AAV9 to newborn Mct8 deficient mice was able to increase T3 in their brain and induce a T3-mediated effect⁵. However, these mice had no neurological deficit owing to an alternative transporter. Recently the same gene therapy was given to peripubertal Mct8 deficient-dKO mice that have neurocognitive abnormalities. It corrected the neurological abnormalities, learning and recall abilities but not the high blood T3 causing the increased metabolism⁶.

A combined gene and DITPA treatment should correct both neuropsychomotor and metabolic defects that each treatment alone could not achieve, providing full rescue of the genetic defect. Thus, there is a need for gene therapy, and especially gene therapy combined with dosing regimens of DITPA, that are effective at treating AHDS and symptoms of ARDS.

### REFERENCES

1. Allan W, Herndon C N, Dudley F C. Some examples of the inheritance of mental deficiency: apparently sex-linked idiocy and microcephaly. Am J Ment Defic. 1944; 48:325-334.
2. Dumitrescu A M, Liao X H, Best T B, Brockmann K, Refetoff S. A Novel Syndrome Combining Thyroid and Neurological Abnormalities Is Associated with Mutations in a Monocarboxylate Transporter Gene. Am J Hum Genet. 2004; 74(1):168-175.
3. Di Cosmo C, Liao X H, Dumitrescu A M, Weiss R E, Refetoff S. A thyroid hormone analogue with reduced dependence on the monocarboxylate transporter 8 (MCT8) for tissue transport. Endocrinology. 2009; 150(9):4450-4458.
4. Verge C F, Konrad D, Cohen M, et al. Diiodothyropropionic Acid (DITPA) in the Treatment of MCT8 Deficiency. J Clin Endocrinol Metab. 2012; 97(12):4515-4523.
5. Iwayama H, Liao X H, Braun L, et al. Adeno Associated Virus 9-Based Gene Therapy Delivers a Functional Monocarboxylate Transporter 8, Improving Thyroid Hormone Availability to the Brain of Mct8-Deficient Mice. Thyroid. 2016; 26(9):1311-1319.
6. Liao X-H, P. A, Shelest O, et al. AAV9-MCT8 delivery at juvenile stage ameliorates neurological and behavioral deficits in an Allan-Herndon-Dudley Syndrome mouse model. 2021.

### DISCLOSURE

The present subject matter is directed to methods of gene therapy for treating a subject with Allan-Herndon-Dudley syndrome, and to methods of combining such gene therapy with administration of 3,5-diiodothyropropionic acid (DITPA) for treating a subject with Allan-Herndon-Dudley syndrome.

### BRIEF DESCRIPTION OF THE DRAWINGS

FIGS. 1A-1B show the study design and human MCT8 expression in the liver and brain regions after IV AAV9-MCT8 injection of P30 dKO mice. FIG. 1A is a schematic of experimental design. dKO mice were treated at postnatal day 30 (P30) by tail vain (IV) delivery of AAV9-MCT8 at a dose of 50×1010 vp/g. FIG. 1B shows the quantification of MCT8 mRNA levels by qRT-PCR showed MCT8 reexpression relative to the three housekeeping genes Polr2a, Actb and Gapdh in the liver and different brain regions of dKO treated animals. One-way ANOVA with Tukey's multiple comparisons. The data are presented as mean, error bars represent SEM. (*P<0.05, **P<0.01, ***P<0.001, ****P<0.0001).

FIGS. 2A-2E show locomotor performance is improved in dKO mice treated at P30. FIG. 2A is a graph demonstrating locomotor deficiencies were monitored by a rotarod test. Data was analyzed with mixed model regression with random intercept and the fixed-factors of time, group, and the interaction term of group with time. To compare learning curves between groups the R coefficients were compared. WT untreated mice (black line, n=16), treated dKO mice (purple line, n=13), untreated dKO mice (grey line, n=24). FIGS. 2B and 2C are plots reflecting open field test of horizontal locomotion (2B) and vertical rearing (2C). FIGS. 2D and 2E reflect paw print assessment at P120 to determine stride length (2D) and hind paw angle (2E). For 2B-2D one-way ANOVA with Tukey's multiple comparisons was used. For 2E, Mann-Whitney non-parametric test for independent samples was used. The data are presented as mean, error bars represent SEM. (*P<0.05, **P<0.01, ***P<0.001, ****P<0.0001).

FIGS. 3A-3D show improved cognitive performance in dKO mice treated at P30. The cognitive-related behavioral performance was assessed at P140. FIGS. 3A-3C: In a Barnes Maze test, the ability of mice to discover and then recall the location of an escape hole was evaluated during the learning phase, at training, day 1-4 (3A), testing on day 7, after a 2-day break (3B), and following reverse re-positioning of the escape hole on days 8 and 9 (3C). The latency to successful location of the escape hole was recorded. Data was analyzed with mixed model regression with random intercept and the fixed-factors of time, group, and the interaction term of group with time. To compare learning curves between groups the R coefficients were compared. FIG. 3D is a plot showing spontaneous alternation between the arms of a Y-maze assessed over a 5-min period. One-way ANOVA with Tukey's multiple comparisons. The data are presented as mean, error bars represent SEM. (*P<0.05, **P<0.01, ***P<0.001, ****P<0.0001). All analyses were double-blind.

FIGS. 4A-4L show brain T3 content and T3-induced gene expression in dKO mice treated at P30. T3 content as measured is shown in the thalamus (4A), hippocampus (4B), and parietal cortex (4C). T3-induced genes were examined by qRT-PCR. Hairless (Hr) expression was measured in the thalamus (4D), hippocampus (4E), and parietal cortex (4F). Expression of Aldehyde dehydrogenase family 1, subfamily A1 (Aldh1a1) was measured in the thalamus (4G), hippocampus (4H), and parietal cortex (4I). Expression of Carbonyl reductase 2 (Cbr2) was measured in the thalamus (4J), hippocampus (4K), and parietal cortex (4L). The data are presented as mean, error bars represent SEM. For FIGS. 4C, 4G and 4J, Mann-Whitney non-parametric test for independent samples was used. For FIGS. 4A, 4B, 4D, 4E, 4F, 4H, 4I, 4K and 4L, one-way ANOVA with Tukey's multiple comparisons was used. The data are presented as mean, error bars represent SEM. (*P<0.05, **P<0.01, ***P<0.001, ****P<0.0001).

FIGS. 5A-5J show liver T3 content, T3-induced gene expression and serum TSH and TH concentrations in dKO mice treated at P30. Livers tissue was obtained for measuring T3 concentrations (5A), and for qRT-PCR analysis of T3-induced gene expression including iodothyronine deiodinase 1 (dio1) (5B), malic enzyme 1 (Me1) (5C), and uncoupling protein 2 (Ucp2) (5D). Concentrations of hormones in serum are shown for TSH (5E), T4 (5F), T3 (5G), and reverse T3 (rT3) (5H). Ratios of T3/rT3 (5I) and T3/T4 (5J) were calculated. Data are presented as mean, error bars represent SEM. For FIG. 5A, no significant changes were found by One-way ANOVA. Student t-test was used to compare every two treatments. For FIGS. 5E, 5H, and 5J, Mann-Whitney non-parametric test for independent samples was used. For FIGS. 5B-5D, 5F, 5G, and 5I, One way ANOVA with Tukey's multiple comparisons was used. The data are presented as mean, error bars represent SEM. (*P<0.05, **P<0.01, ***P<0.001, ****P<0.0001).

FIG. 6 is a graph of the dose response for DITPA administration to liver in vitro, and its effect on D1 enzymatic activity, i.e., converting T₄ to T₃.

FIG. 7 shows the effect of AAV9-hMCT8 treatment of dKO mice (dKO Rxed). T₃ content and response to of a TH regulated gene (Aldh1a1) in two brain areas.

FIG. 8 is a schematic representation of the treatment protocol.

### DESCRIPTION OF EMBODIMENTS

### I. Gene Therapy

Background: Allan-Herndon-Dudley syndrome (AHDS) is a severe psychomotor disability disorder that also manifests characteristic abnormal thyroid hormone (TH) levels. AHDS is caused by inactivating mutations in monocarboxylate transporter 8 (MCT8), a specific TH plasma membrane transporter widely expressed in the central nervous system (CNS). MCT8 mutations cause impaired transport of TH across brain barriers, leading to insufficient neural TH supply. There is currently no successful therapy for the neurological symptoms. Earlier work has shown that intravenous, but not intracerebroventricular adeno-associated virus serotype 9 (AAV9)-based gene therapy given to newborn Mct8 knockout (Mct8−/y) male mice increased T3 brain content and partially rescued TH-dependent gene expression, suggesting a promising approach to treat this neurological disorder.

Methods: The potential of intravenous delivery of AAV9 carrying human MCT8 was tested in the well-established Mct8−/y/Organic anion-transporting polypeptide 1c1 (Oatp1c1)−/− double knockout (dKO) mouse model of ARDS, which unlike MCT8−/y mice, displays both neurological and TH phenotype. Further, as the condition is usually diagnosed during childhood, treatment was given intravenously to P30 mice and psychomotor tests were carried out blindly at P120-P140 after which tissues were collected and analyzed.

Results: Systemic intravenous delivery of AAV9-MCT8 at a juvenile stage led to improved locomotor and cognitive function at P120-P140, which was accompanied by a near normalization of T3 content and an increased response of positively regulated TH-dependent gene expression in different brain regions examined (thalamus, hippocampus, and parietal cortex). Effects on serum TH concentrations and peripheral tissues were less pronounced, showing only improvement in the serum T3/rT3 ratio and in liver deiodinase 1 expression.

Conclusion: Intravenous administration of AAV9, carrying the human MCT8, to juvenile dKO mice manifesting ARDS has long-term beneficial effect predominantly on the CNS. This preclinical study indicates that this gene therapy ameliorates the devastating neurological symptoms in patients with ARDS.

In one embodiment, the present technology is directed to methods of using gene therapy to treat and correct neurological abnormalities associated with ARDS in a subject in need of such treatment, wherein the gene therapy comprises administration of AAV9-MCT8 to the subject, wherein the administration preferably is intravenous.

### Introduction

Thyroid hormones (THs) are essential for the development and metabolic homeostasis of most organs and tissues (1). The major form of TH released in the blood from the thyroid gland is thyroxine (T4), which acts as a prohormone. T4 conversion to the active hormone, triiodothyronine (T3), or to the inactive form, reverse T3 (rT3), takes place intracellularly by iodothyronine deiodinases enzymes (2). The main mechanism of T3 action is achieved through binding to specific nuclear receptors, which in turn operate as regulators of gene transcription (3). Since TH metabolism and action are intracellular events, they require the presence of TH specific transporters mediating cellular TH uptake and efflux (4). The solute carrier family 16, member 2 (SLC16A2) gene, located on the X-chromosome, encodes for the monocarboxylate transporter 8 (MCT8) protein (5). MCT8 is well conserved throughout vertebrate evolution and is widely expressed in the body and central nervous system (CNS) (6). A key function of MCT8 is to facilitate TH transport across plasma membranes (5).

Inactivating MCT8 gene mutations in males cause a severe form of psychomotor disability (7-9), clinically described by Allan Herndon Dudley Syndrome (ARDS) (10). Patients exhibit neurological impairments including severe intellectual disability, truncal hypotonia, dystonia and movement disorders. MCT8-deficiency also causes a TH phenotype, including elevated serum T3 levels, low rT3 and T4 with normal or slightly elevated thyroid stimulating hormone (TSH) resulting in markedly elevated free T3/T4 and T3/rT3 ratios (11).

Two independently generated Mct8-KO mouse models (12, 13) closely recapitulate the TH phenotype observed in patients with ARDS, but do not display expected neurological or behavioral phenotypes. This is due to a milder TH deprivation in mouse brains owing to a T4-specific transporter not present in the human blood brain barrier (BBB). Specifically, the Organic anion-transporting polypeptide 1c1 (Oatp1c1), encoded by the slco1c1 gene, was identified in mice, but not human, brain capillaries (14-16). Double knockout mct8^{-/y}; oatp1c1^{-/-} mice (dKO) display disease-relevant phenotypes including an impaired TH transport into the CNS and consequently a significantly decreased number of cortical parvalbumin-positive GABAergic interneurons, reduced myelination and pronounced locomotor abnormalities (17). These results indicate that in mice Mct8 (together with Oatp1c1) plays a crucial role in the transport of THs into the CNS and, importantly, provides a robust disease model for human MCT8-deficiency (18, 19). To progress from an animal model to a human-based model, induced pluripotent stem cells (iPSCs) were derived from ARDS patients and differentiated into brain microvascular endothelial-cells, which showed MCT8-dependent transport of THs across the human BBB (16, 20). However, MCT8 is not restricted to the brain endothelium, and it also affects TH transport across neural cell plasma membranes (21).

Gene therapy offers a promising approach to treat monogenic disorders. Spinal muscular atrophy type 1 patients, carrying deleterious mutations in the SMN1 gene, were treated by a single intravenous infusion of adeno-associated virus serotype 9 (AAV9) containing DNA coding for SMN, resulting in improved survival, as well as achievement of motor milestones and motor functions (22, 23), with subsequent FDA approval of the ZOLGENSMA gene therapy product. Additional examples for beneficial gene therapy approach for monogenic disorders have been reported recently (24-26).

While intracerebroventricular (ICV)-delivery directly targets the brain ventricles, thereby circumventing the blood brain barrier (BBB), intravenous (IV)-delivery offers systemic delivery, transducing primarily tissues outside the CNS including blood vessels. Importantly, it has been shown that AAV9 IV-delivery can cross the BBB and efficiently infect CNS cells (27). In a recent proof of concept study, an AAV9-MCT8 construct was delivered by ICV or IV injections into neonatal Mct8-KO (MCT8^{-/y}) mice (28), with an increase in brain TH signaling upon IV, but not ICV, delivery. However, since the Mct8-KO mice do not display neurological impairments, it is unclear whether this approach results in rescue of the neurological symptoms.

Here, we tested the potential of IV delivery of AAV9-MCT8 in dKO mice. We chose to treat juvenile male mice at postnatal day 30 (P30) and tested the potential rescue of neurological and behavioral parameters at adulthood. This approach was espoused as the diagnosis of MCT8 deficiency is usually made in childhood.

Materials and Methods

All procedures were approved by Cedars-Sinai Medical Center's Institutional Animal Care and Use Committee (IACUC #009128). mct8^{-/-}/oatp1c1^{-/-} females and mct8^{-/y}/oatp1c1^{-/-} males with C57BL/6 background were paired to generate dKO pups. WT C57BL/6 were used as controls. Only males were selected for all treatments. AAV9-MCT8 was administered to P30 juvenile dKO mice and controls by tail vein injection containing 50×1010 viral particles vp/g in a volume of 20 μl/g. Behavioral as well as biochemical and molecular measurements were all performed on tissues and serum at P120-P140 and analyzed double blinded without the knowledge to which group the mice belonged. Mice identities were blinded by the person administering the AAAV9-MCT8 and were unknown to the technicians while performing the behavioral assays, dissection, tissue collection and biochemical analysis. This was unblinded when results were assembled and for the statistical analysis. Animals were subjected to behavioral analysis before being sacrificed for tissue collection. Thus, results from the same mice are presented in all figures.

Statistical Analysis

All scatter plots were first tested for their normal distribution using Kolmogorov-Smirnov test, with the Dallal-Wilkinson-Lillie for corrected P value. Data sets that were normally distributed were tested using one-way ANOVA with Tukey's post-hoc test for multiple comparisons. Data sets that were not normally distributed were compared using Mann-Whitney non-parametric test for independent samples. Behavior data collected over time including the rotarod test and Barnes maze were analyzed with mixed model regression with random intercept and the fixed-factors of time (as a continuous variable), treatment group, and the interaction term of group with time. To compare learning curves between groups, the β coefficients were compared and differences were considered significant at the step-down Bonferroni-corrected alpha level of <0.05. Residuals were inspected to confirm the fit of the modeling. Data analysis was performed with GraphPad Prism v8.0.0 and SAS Enterprise Guide v8.2. All data are presented as mean±SEM. p<0.05 was considered statistically significant. *P<0.05, **P<0.01, ***P<0.001, ****P<0.0001.

Results

IV delivery of AAV9-MCT8 at P30 improves locomotor performance of dKO mice.

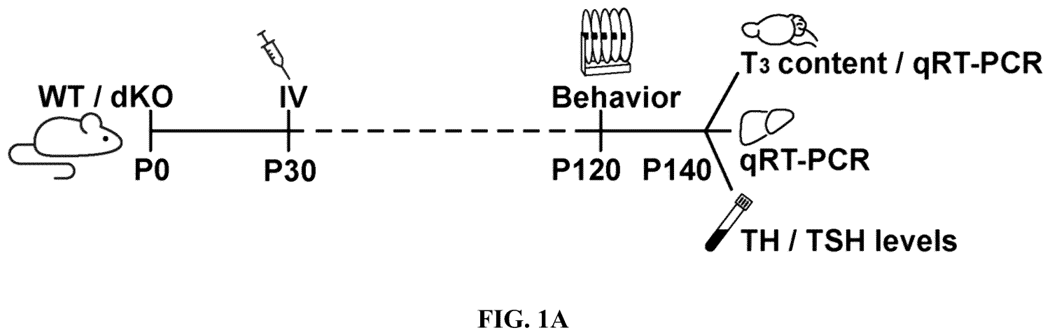

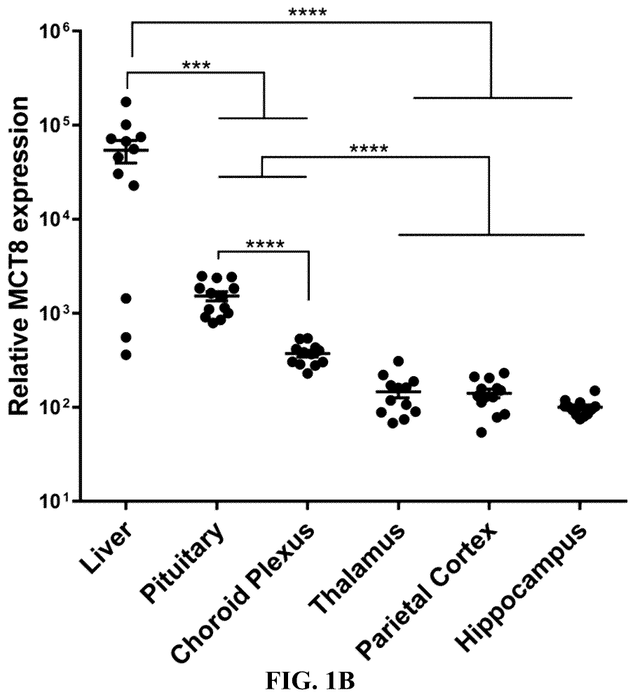

Given that patients with AHDS are often diagnosed during childhood, treatment feasibility should optimally be tested at a juvenile stage. Previous experience (28) indicated that IV delivery is more feasible compared to ICV delivery, as IV is a simpler route and would target not only the brain but other body regions that express MCT8 such as the liver. Therefore, we tested IV delivery at the previously tested dose of 50×1010 vp/g of AAV9-MCT8 (28) to peri-pubertal P30 dKO mice (FIG. 1A).

In order to confirm that the viral delivery resulted in expression of human MCT8, liver and brain were collected at P140. As expected, human MCT8 mRNA expression was not detected in wild type (WT) or dKO untreated mice (data not shown). MCT8 expression was observed in the liver and various brain regions of AAV9-MCT8 IV-treated dKO mice, with the highest levels seen in the liver. Pituitary and choroid plexus had higher MCT8 levels compared to the BBB-protected regions of the thalamus, parietal cortex, and hippocampus (FIG. 1B).

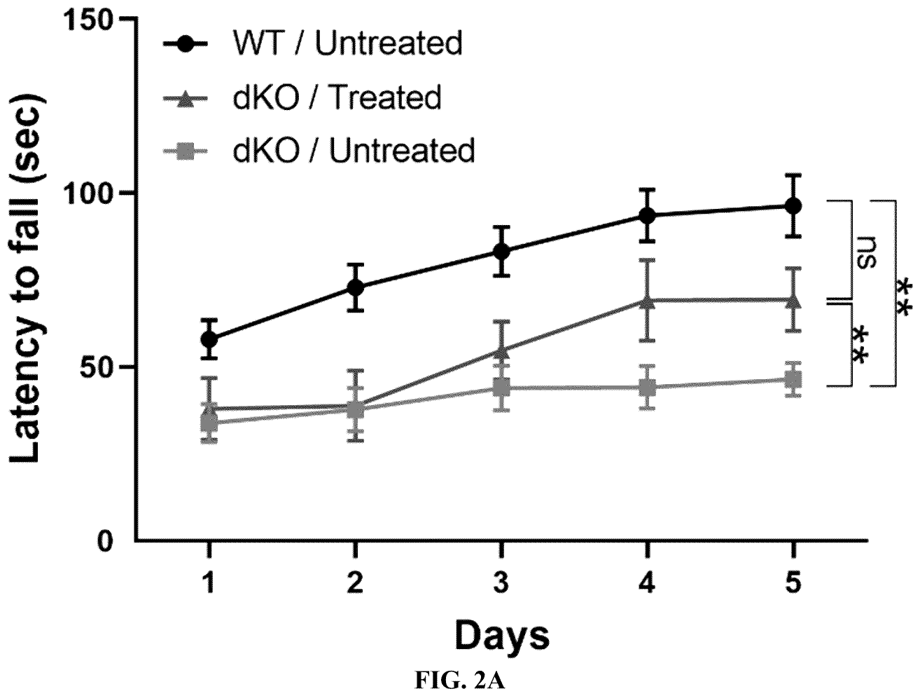

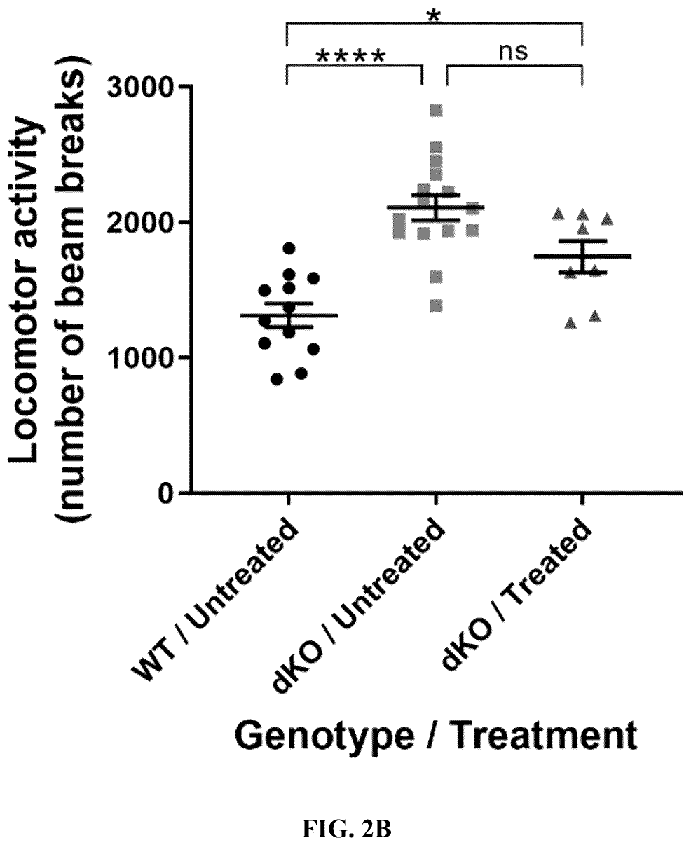

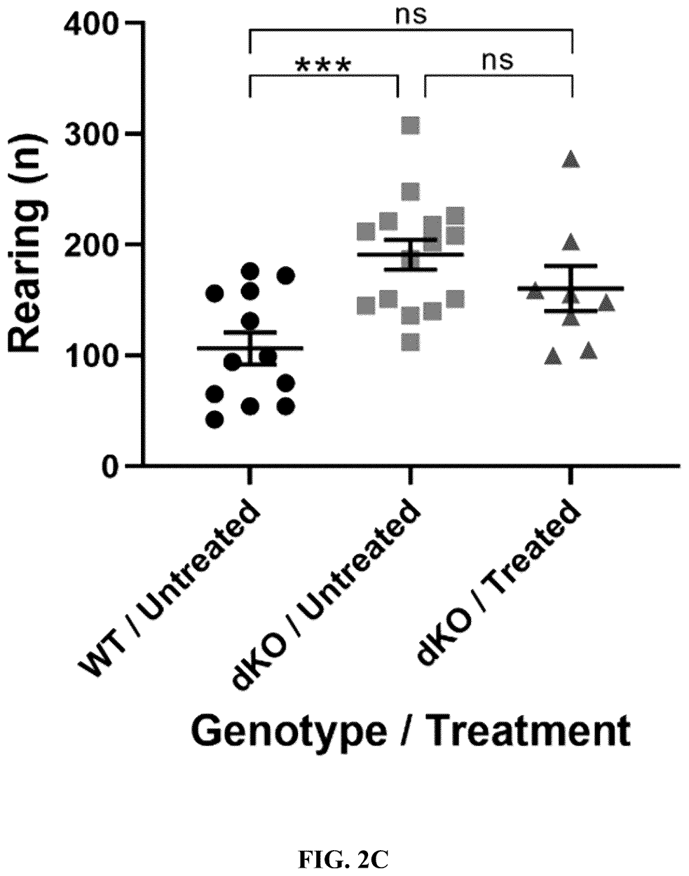

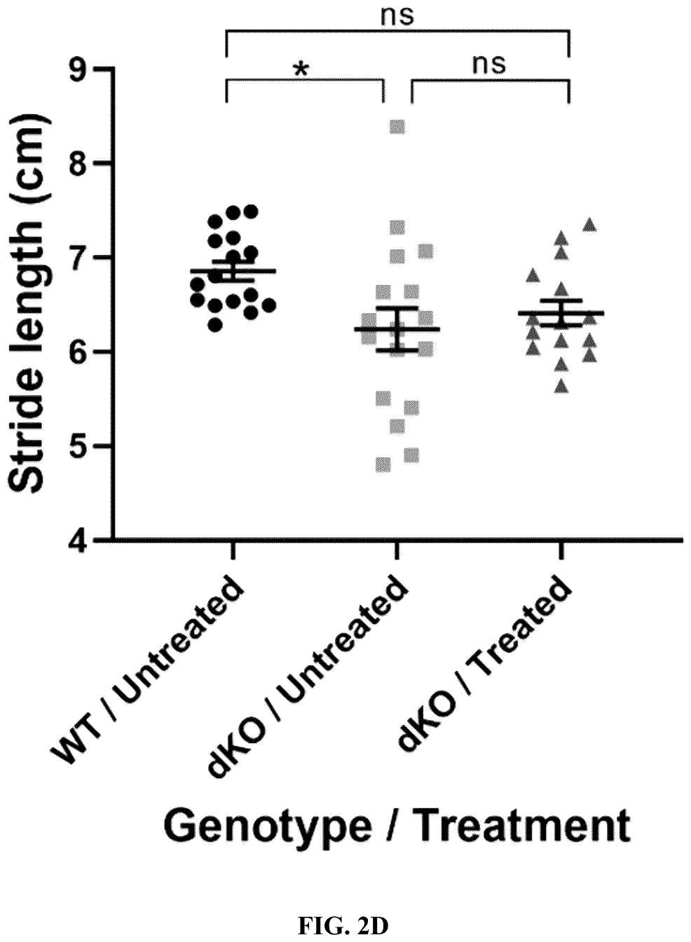

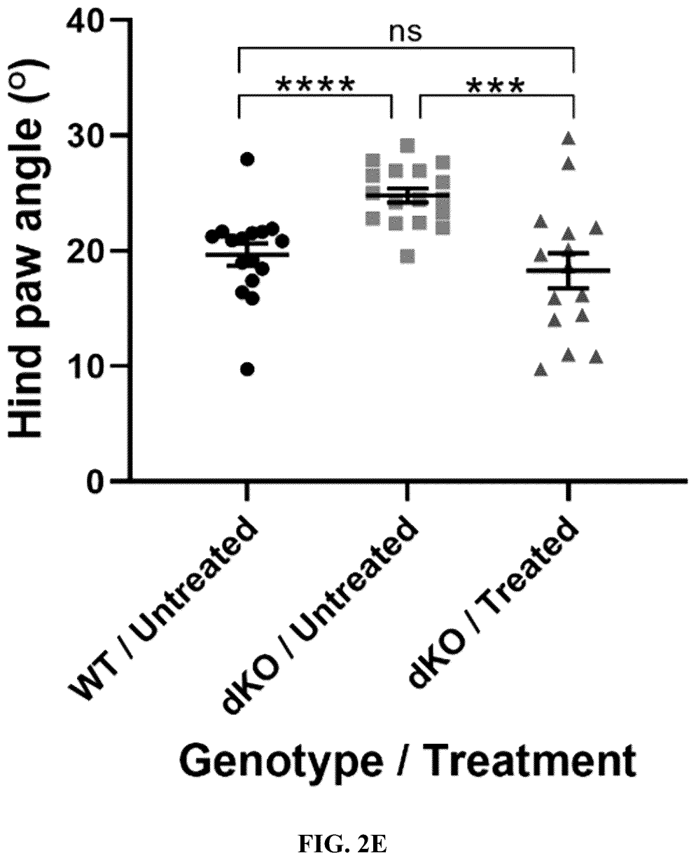

Mice underwent various behavioral analyses to assess locomotor function, which were performed at P120, when differences between WT and dKO were previously reported (29). Assessment by a rotarod test showed that untreated dKO mice had an overall reduced latency to fall and decreased learning curve compared to the WT group (FIG. 2A). Following IV delivery of AAV9-MCT8, dKO mice showed a significant increase in the latency to fall and an increased learning curve, indicating that treatment improved locomotor performance and potentially cognitive function. Overall locomotor activity assessed by an open field test as well as rearing behavior were both significantly higher in untreated dKO mice compared to WT mice, and did not significantly change following treatment (FIGS. 2B and 2C). Finally, hind paw analysis showed that there was no significant improvement in the stride length and a complete recovery in the hind paw angle (FIGS. 2D and 2E). Collectively data demonstrate that AAV9-MCT8 treatment at P30 provided recovery in some, but not all locomotion parameters in dKO mice.

IV delivery of AAV9-MCT8 at P30 improves cognitive performance in dKO mice.

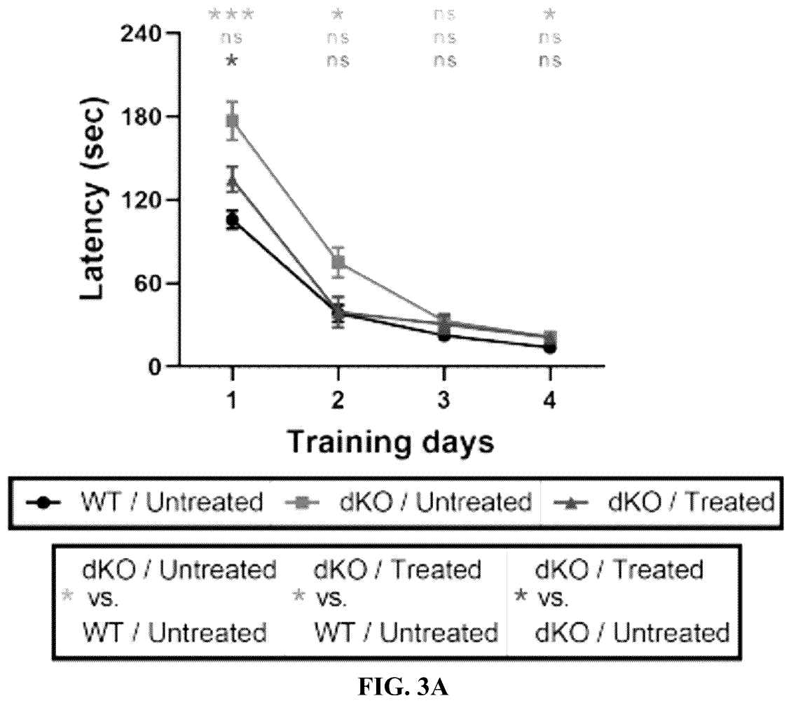

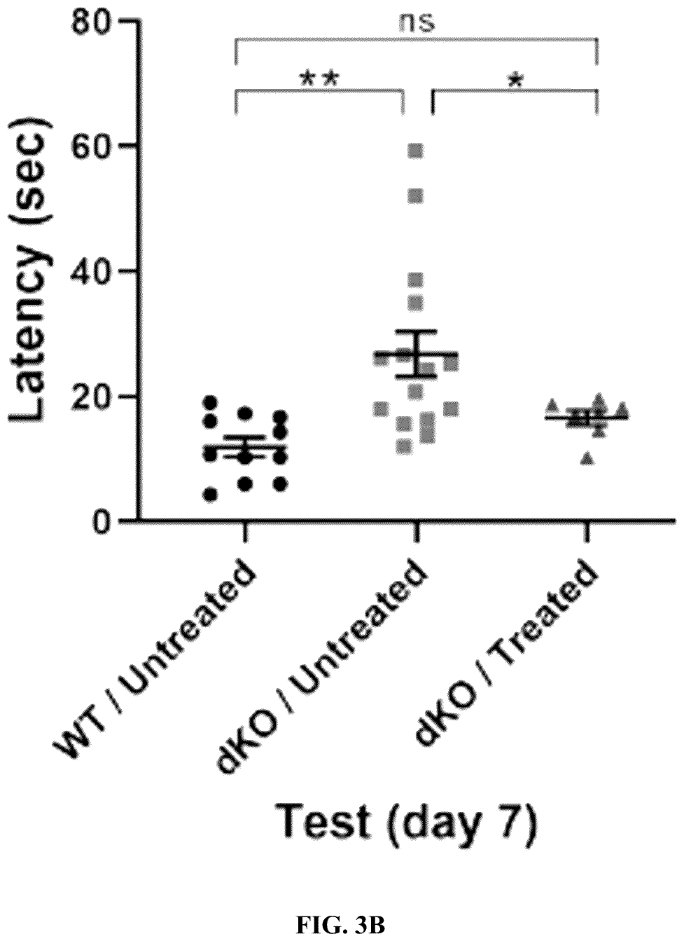

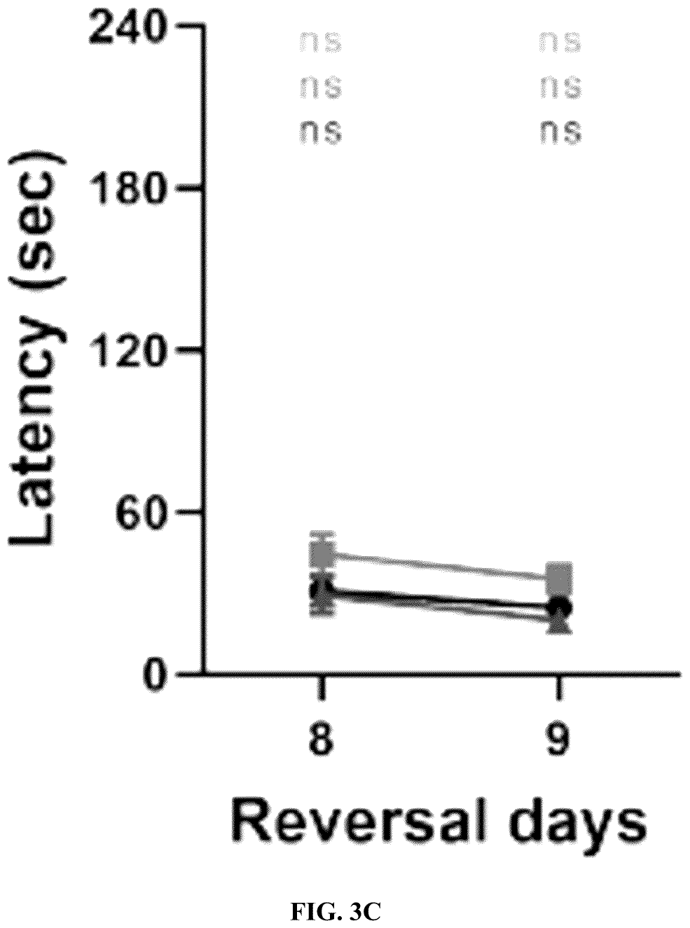

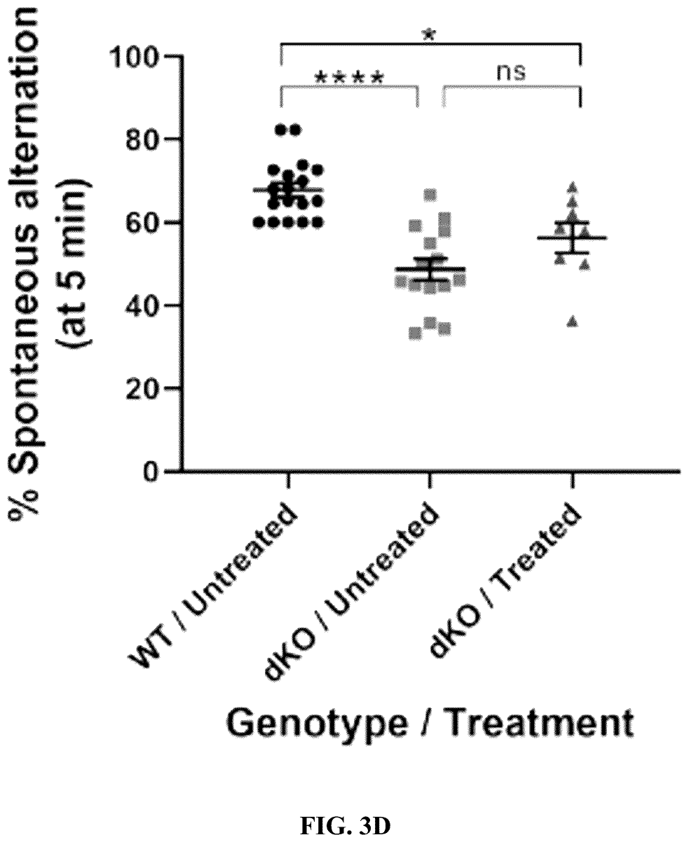

Learning, spatial memory and memory recall were assessed using a Barnes maze (30). During the 4-day training period, treatment did not significantly improve the learning curve (FIG. 3A). Following a 2-day break, untreated dKO mice required higher latency than untreated WT and treated dKO mice (FIG. 3B). The position of the escape hole was then moved during two days of training during which no significant differences were observed between all groups (FIG. 3C). These data suggest that the AAV9-MCT8 IV treatment of dKO mice at P30 resulted in a partial rescue in the learning and recall ability.

A spontaneous alternation maze (Y-maze) test was used to further examine spatial memory. Untreated dKO mice demonstrated a significant decrease in the percent of spontaneous alterations compared to untreated WT mice, which was not significantly improved in the AAV9-MCT8-treated dKO mice (FIG. 3D). The results of both tests suggest that IV-treatment of P30 dKO mice with AAV9-MCT8 improves cognitive performance as well as restores some spatial and learning memory.

IV delivery of AAV9-MCT8 at P30 partially restores brain T3-content in dKO mice.

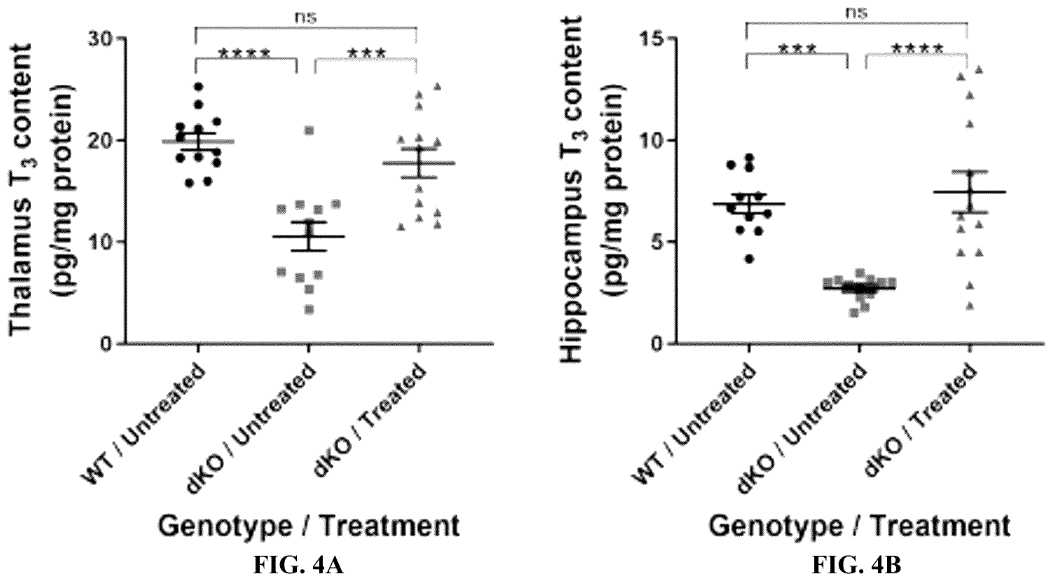

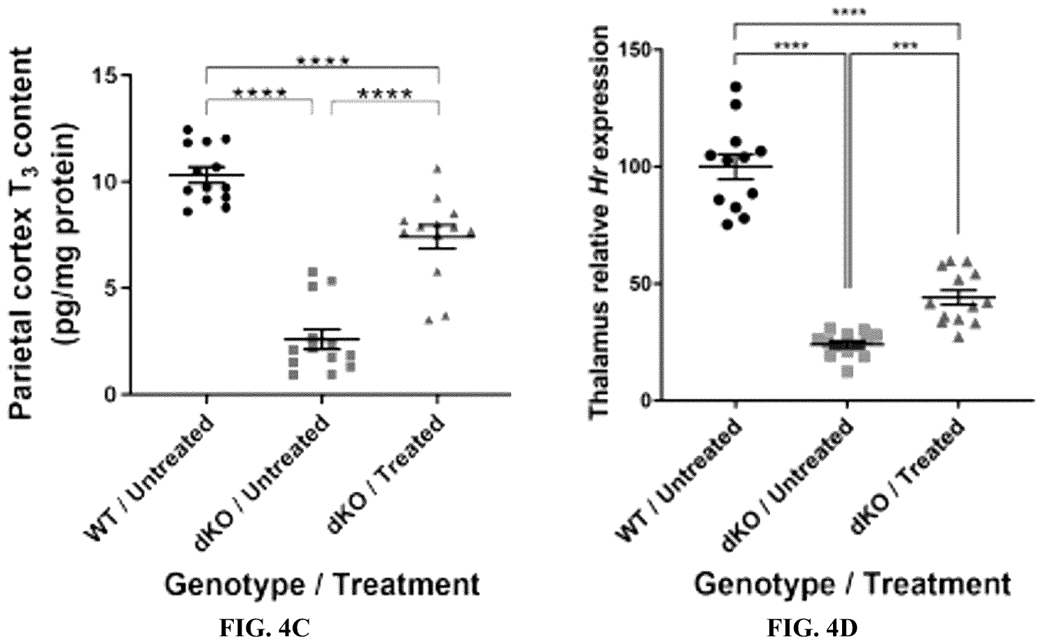

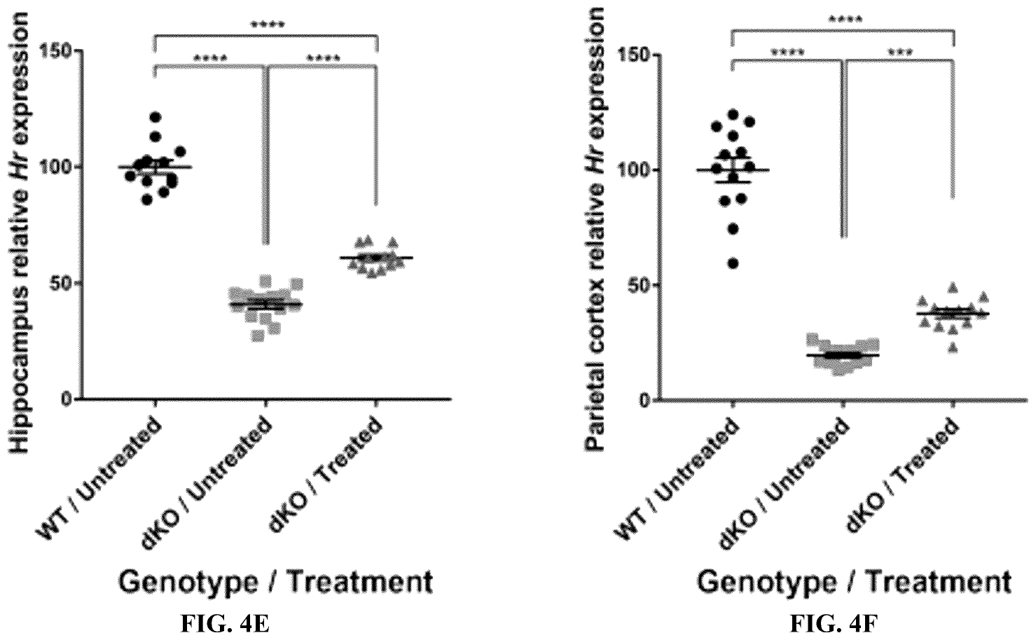

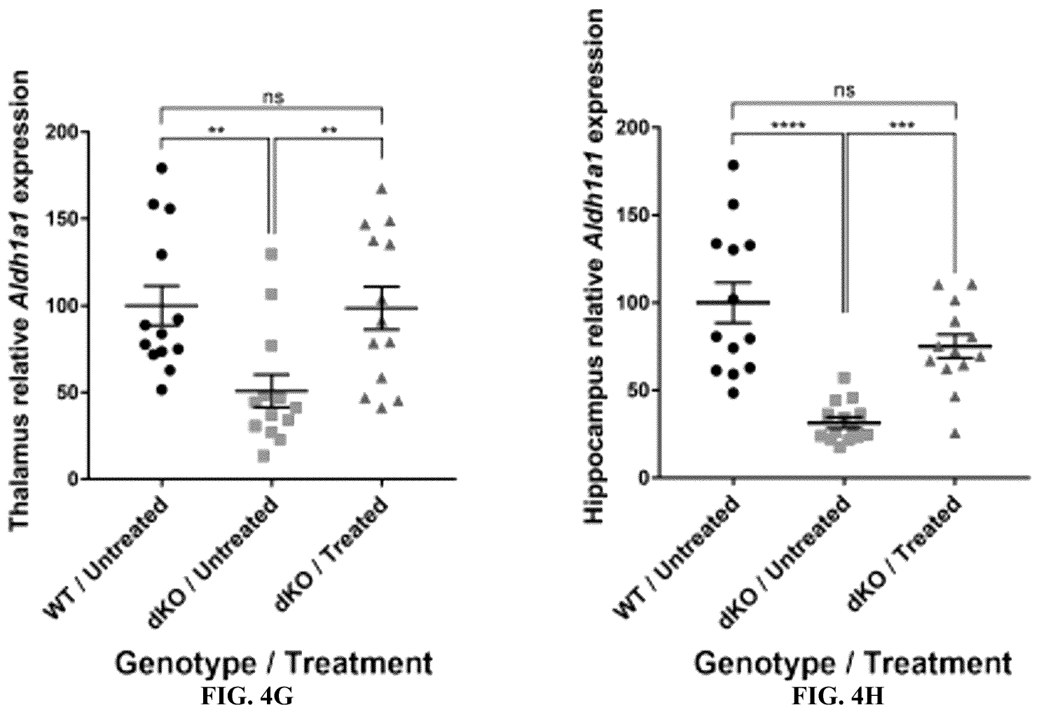

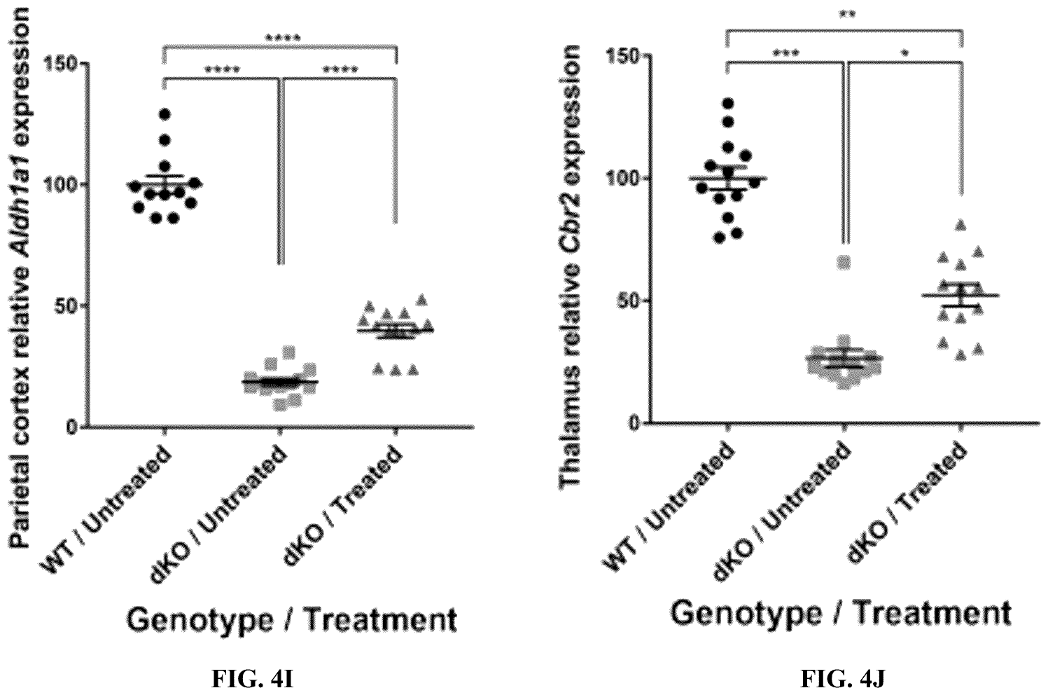

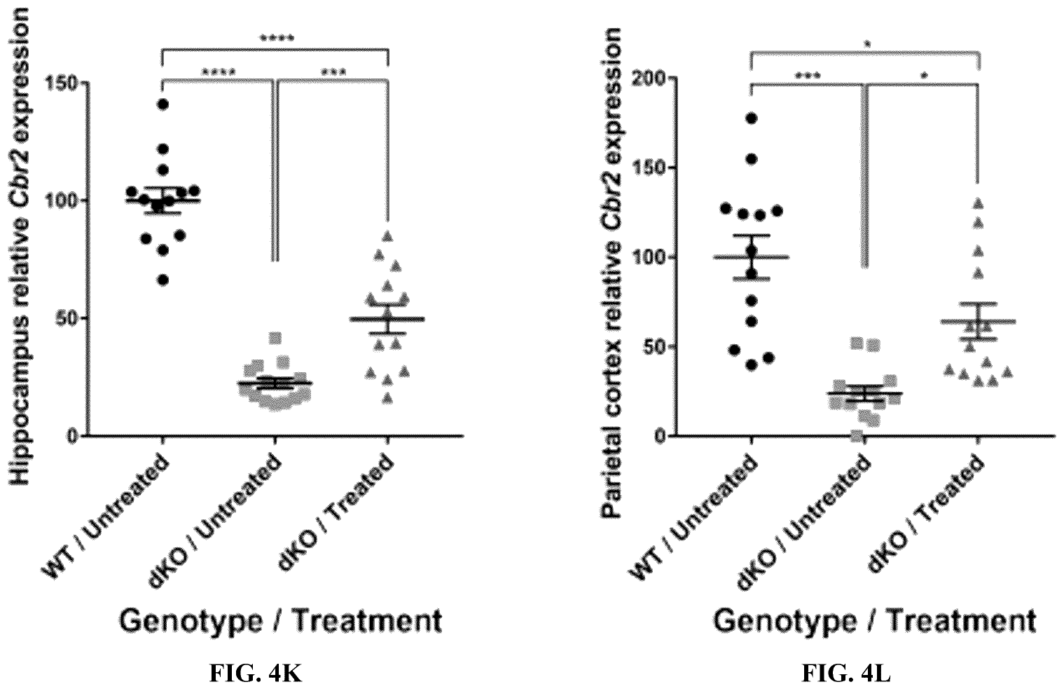

In order to assess effects on pathophysiology, the serum, liver and brain were collected at P140 (FIG. 1A). Examining the T3 brain content, we showed that T3 levels in the thalamus (FIG. 4A) and hippocampus (FIG. 4B) of treated dKO mice were fully normalized reaching WT levels. A significant increase in brain T3 content was also observed in the parietal cortex (FIG. 4C). Given the low brain levels of MCT8 expression following treatment, these results suggest that low MCT8 expression (FIG. 1B) is sufficient to normalize brain T3 content.

IV delivery of AAV9-MCT8 at P30 corrects T3-inducible gene expression.

To assess the T3-effect in the different brain regions, we next studied T3-inducible gene expression by quantitative real-time polymerase chain reaction (qRT-PCR). These genes were selected based on their known response to T3 (31). Hairless (Hr) levels were significantly improved in the thalamus (FIG. 4D), the hippocampus (FIG. 4E) and parietal cortex (FIG. 4F) of treated dKO mice. Aldehyde dehydrogenase 1 family member a1 (Aldh1a1) levels were fully rescued in the thalamus (FIG. 4G) and hippocampus (FIG. 4H) and significantly improved in the parietal cortex (FIG. 4I). Finally, Carbonyl reductase 2 (Cbr2) levels were also significantly improved in treated dKO compared to untreated dKO mice in the thalamus, the hippocampus and the parietal cortex (FIG. 4J-L). These results suggest that IV delivery of AAV9-MCT8 to P30 dKO mice can substantially improve and maintain long-term brain T3 content and T3-inducible gene expression.

IV delivery of AAV9-MCT8 at P30 partially rescues phenotypes in the liver and minimally in serum of dKO mice.

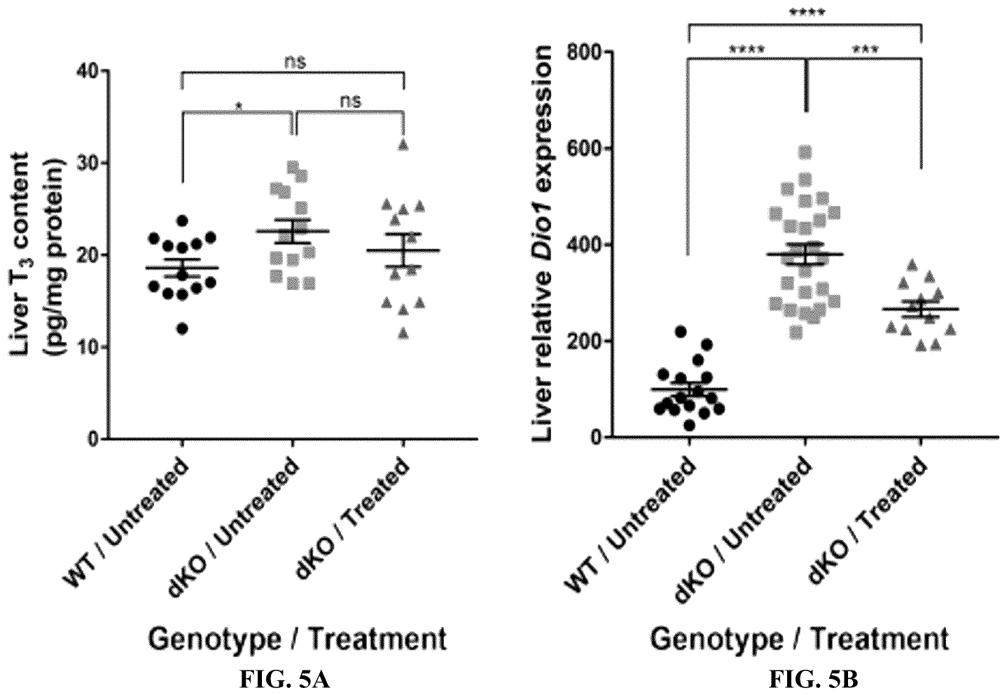

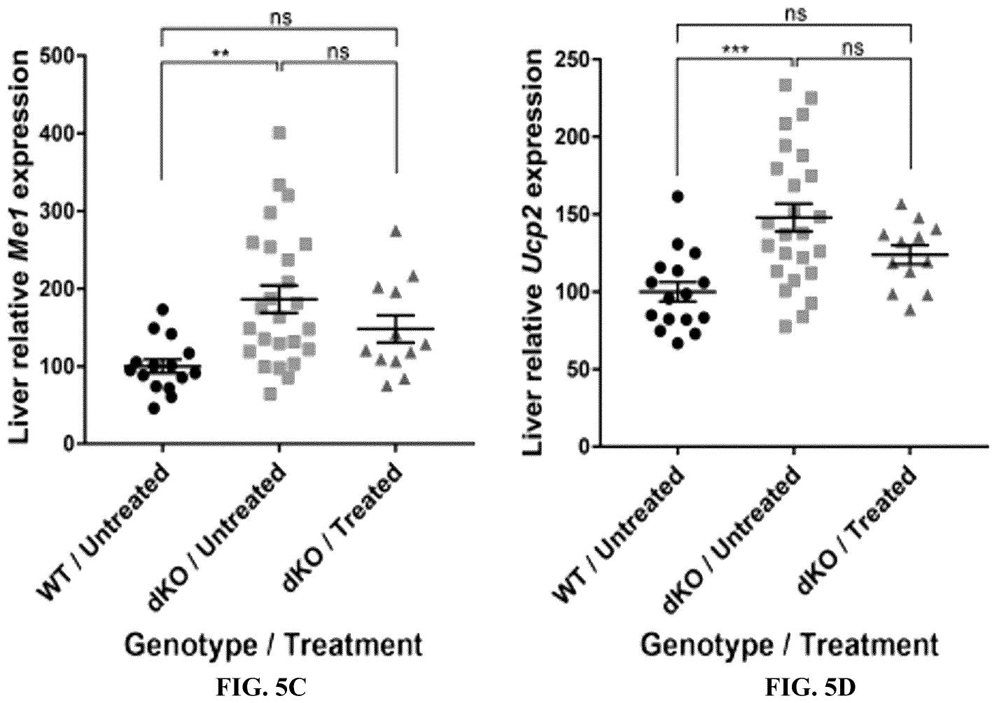

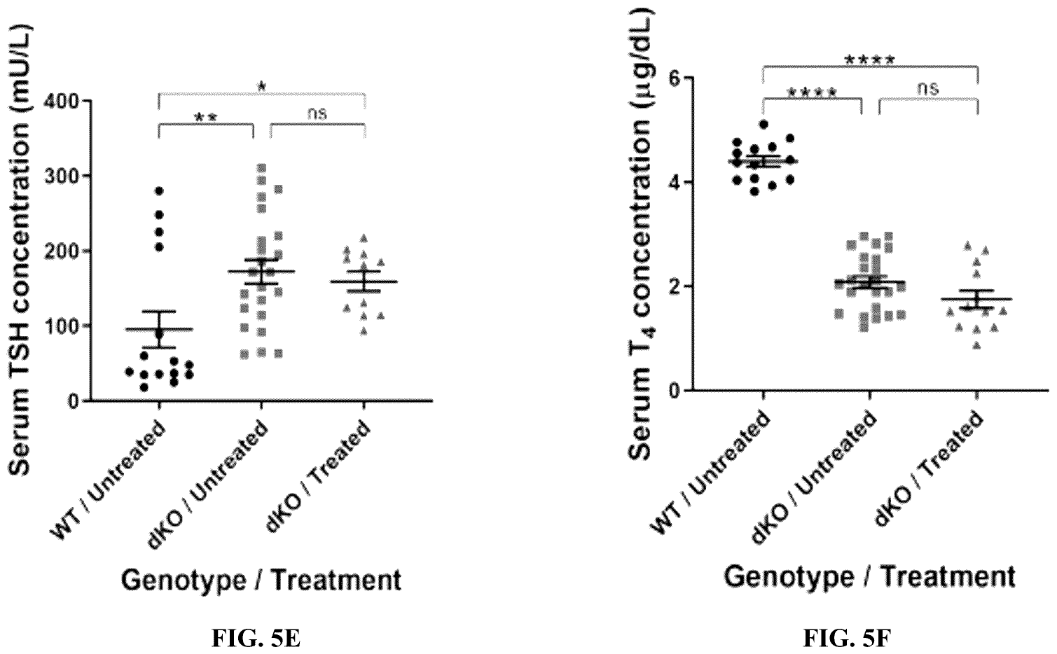

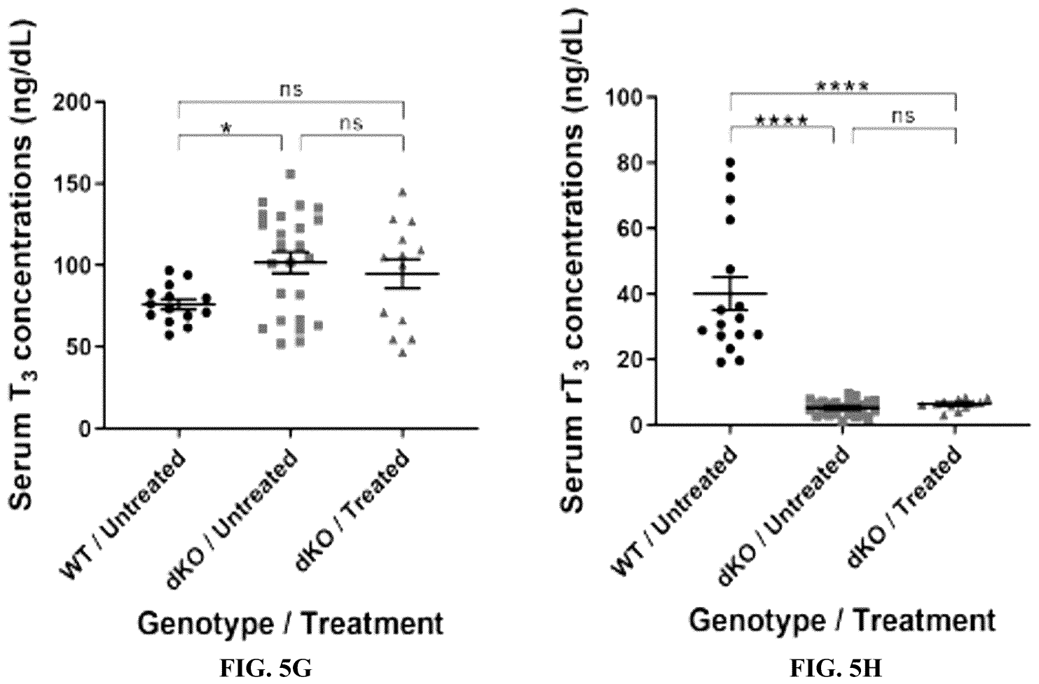

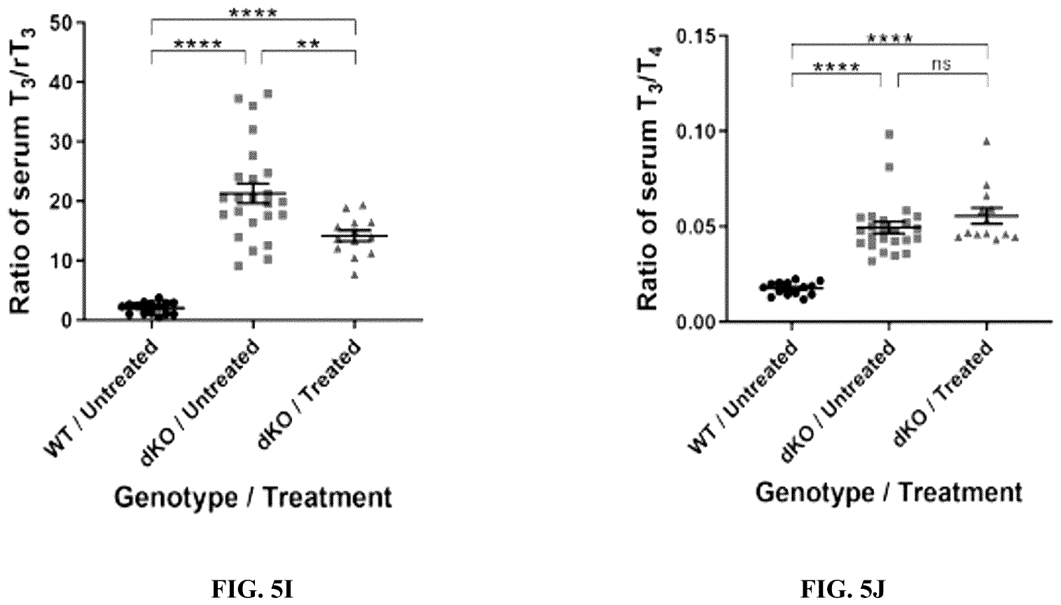

The significantly higher level of MCT8 expression in the liver (FIG. 1B) is expected with IV delivery, which can easily penetrate the blood vessels of the liver compared to the BBB capillaries (32). In contrast to the brain TH deficiency, patients with AHDS experience TH excess in peripheral tissues caused by the high serum T3 levels. We therefore measured liver T3 levels (FIG. 5A). The treatment did not result in a significant decrease of T3 levels in treated dKO mice. Analysis of liver T3-inducible genes was performed by qRT-PCR. The expression levels of deiodinase 1 (Dio1), malic enzyme 1 (Me1) and uncoupling protein 2 (Ucp2) (FIG. 5B-D), and the liver T3 level were all significantly elevated in the dKO untreated group compared to their WT littermates, confirming the effect of TH excess in liver. AAV9-MCT8 delivery led to a significant decrease in Dio1 mRNA levels in treated dKO mice compared to untreated dKO mice. However, there was no significant reduction in Me1 and Ucp2 levels in response to treatment.

Patients with AHDS have abnormal serum TH levels, including elevated T3, low rT3 and T4 with normal or slightly elevated TSH, resulting in low T3/T4 and T3/rT3 ratios. The increased liver deiodinase 1 enzymatic activity is one of the mechanisms responsible for these serum thyroid tests and a decrease in its expression is needed to ameliorate this phenotype (33). In order to test the effect of IV delivery of AAV9-MCT8 at P30 on the serum TH phenotype in dKO mice, blood was collected from P140 mice and serum TSH and TH levels were quantified. Serum levels of TSH, T4, T3, and rT3 as well as the T3/rT3 and the T3/T4 ratios were all significantly altered in dKO mice compared to their WT littermates (FIG. 5E-J). While AAV9-MCT8 treatment did not significantly alter serum levels of TSH, T4, T3, rT3 and T3/T4 ratio, the combination of slight reduction in T3 and increase in rT3 resulted in a significant decrease of the T3/rT3 ratio in agreement with the observed attenuation of Diol expression. This indicates that AAV9-MCT8 delivery at a juvenile stage can partially improve the abnormalities in serum.

### DISCUSSION

In this study, we tested the potential of IV delivery of AAV9-MCT8 to juvenile dKO mice. Our analysis shows long-term expression of human MCT8 within the CNS and in peripheral tissue, suggesting that AAV9 can efficiently transduce cells with MCT8 in peripheral tissue and within the CNS. Re-expression of MCT8 resulted in improved locomotor and cognitive behavior as well as substantial rescue of T3 content and associated gene expression in different areas of the brain.

MCT8-deficient patients suffer from a severe neuro-psychomotor phenotype and TH excess in peripheral tissues. Thus, an effective therapeutic strategy should account for deficient transport of THs across brain barriers and neural plasma membranes as well as the excess of TH in peripheral tissues. Being a rare disorder, ARDS is often misdiagnosed resulting in later identification of the disease. Moreover, there are currently about 300 diagnosed cases, which mostly involve older children (11, 34). Thus, it is important to develop therapeutics that are effective at juvenile ages. We therefore tested the effect of tail vein IV delivery at P30, which is peri-pubertal when pathophysiological symptoms are apparent in both dKO mice and patients (17, 35, 36).

Endogenously, MCT8 is ubiquitously expressed and is prominently localized in the thyroid, liver, kidneys and CNS (6, 12, 13). In the current study, we showed that IV-administration of AAV9-MCT8 to P30 dKO mice led to long-term MCT8 expression in the CNS and liver.

In the brain, MCT8 expression was observed in various regions, confirming the ability of AAV9 vectors to cross brain barriers and efficiently transduce brain cells during this period of development (27). Interestingly, higher expression was observed in brain regions that are not protected by the BBB such as the choroid plexus and pituitary (37, 38) compared to the thalamus, hippocampus and cortex. Strikingly, in the current study, this long-term brain expression resulted in a nearly complete normalization of T3 brain concentrations and associated gene expressions in the thalamus, hippocampus and parietal cortex. MCT8-deficiency was previously suggested to be caused both, by reduced transport of T3 across brain barriers and across neural cell membranes (16, 21, 29, 39). These results therefore suggest that a rescue of the T3 brain content was achieved. However, future work is required to distinguish whether the improved brain content is caused by MCT8 expression in brain blood vessels or the choroid plexus, which can both serve as gateways to the brain.

The improved brain content was accompanied by improved performance in the rotarod test and in gait analyses. While it remains unclear why other locomotor functions did not change significantly, the observed improvements can be attributed to ameliorations in psychomotor functions. Treated animals also showed an improvement in the learning curve using the Rotarod test, suggesting that treatment may have beneficial effects on cognitive and motor functions. Exploratory behavior, learning and memory are thought to originate in the hippocampus (40). Notably, the mild rescue of hippocampus-dependent learning and memory in the Barnes maze test in response to treatment was observed to be correlated with the significant increase of T3 levels and T3-induced gene expression in the hippocampus.

MCT8 expression was significantly higher in the liver than in the brain. However, no significant rescue was observed in T3 levels in the liver. Nevertheless, a reduction was observed in liver Diol expression, a TH-regulated enzyme that generates T3 from T4 and is responsible in part for the T3 excess in serum (33). This effect on Diol expression resulted in a partial amelioration in serum T3/rT3 ratio, while other parameters were not significantly improved. These results suggest a mild beneficial effect of systemic (IV) delivery of MCT8 on the peripheral tissues. Additional mechanisms contribute to the characteristic serum thyroid tests of AHDS, including decreased thyroidal secretion and altered negative feedback to the hypothalamus and pituitary, and thus have different TH availability (41-43). Thus, to augment the partial rescue, additional TH-normalizing treatments should be considered in conjunction with gene therapy.

There are several current treatment strategies that focus on TH analogs. These thyromimetic compounds are required to activate TH-induced transcriptional pathways via thyroid nuclear receptors, and need to penetrate plasma membranes independent of MCT8. In animal models, the TH analogs DITPA (44, 45), TRIAC (18), TETRAC (19) and Sobetirome (46) were able to restore some of the peripheral and central abnormalities; however, their effects on neurological symptoms remained limited or unknown due to the use of mice that were not neurologically affected. In patients with ARDS, DITPA (47) and TRIAC (48) reduced the high serum T3 concentration, however, there was no evidence for improvement in neurological symptoms. Thus, TH analogs can be considered to be used in combination with gene therapy.

Chemical and pharmaceutical chaperons have also been suggested as an alternative approach. These chaperons can restore the ability of some MCT8 gene mutations to transport TH across plasma membranes in animal models (49, 50); however, their clinical effect has not been assessed to date. Moreover, the use of chemical chaperons is limited only to a handful of missense mutations, and is therefore not applicable to the majority of patients.

Gene therapy, which emerged as a promising approach to treat monogenic developmental neurological disorders (23, 25, 26, 51-53), can overcome these limitations and potentially target all mutations and patients. Moreover, restoration of a functional MCT8 has the potential to resolve TH transport, as well as unidentified MCT8 roles, such as the transport of additional potential substrates.

Although this study shows a promising therapeutic direction, it has limitations. While the dKO mice provide a useful model for ARDS, the symptoms are less severe than in patients. Furthermore, additional disease relevant features such as lack of language or the predisposition to death could not be tackled in this study due to limitations of the model.

Overall, this study shows that IV administration of AAV9-MCT8 to dKO mice provides substantial rescue of molecular and biochemical parameters in the brain, as well as amelioration of the TH excess effect in peripheral tissues. In addition, this treatment improves locomotor and behavioral performance. These findings support future clinical examination of AAV-based MCT8 gene therapy in patients with ARDS.

### REFERENCES

1. Yen P M 2001 Physiological and molecular basis of Thyroid hormone action. Physiol Rev. 81: 1097-1142
2. Gereben B, Zavacki A M, Ribich S, Kim B W, Huang S A, Simonides W S, Zeold A and Bianco A C. 2008 Cellular and molecular basis of deiodinase-regulated thyroid hormone signaling. Endocr Rev. 29:898-938
3. Harvey C B, Williams G R 2002 Mechanism of thyroid hormone action. Thyroid 12:441-446
4. Groeneweg S, Van Geest F S, Peeters R P, Heuer H and Visser W E. 2019 Thyroid Hormone Transporters. Endocr Rev. 41:1-55
5. Friesema E C H, Ganguly S, Abdalla A, Manning Fox J E, Halestrap A P and Visser T J. 2003 Identification of monocarboxylate transporter 8 as a specific thyroid hormone transporter. J Biol Chem 41:40128-40135
6. Vatine G D, Zada D, Lerer-Goldshtein T, Tovin A, Malkinson G, Yaniv K and Appelbaum L. 2013 Zebrafish as a model for monocarboxyl transporter 8-deficiency. J Biol Chem 288:169-180
7. Friesema E C H, Grueters P A, Biebermann H, Krude H, von Moers A, Reeser M, Barrett T G, Mancilla E E, Svensson J, Kester M H A, Kuiper G G J M, Balkassmi S, Uitterlinden A G, Koehrle J, Rodien P, Halestrap A P and Visser T J. 2004 Association between mutations in a thyroid hormone transporter and severe X-linked psychomotor retardation. Lancet 364: 1435-1437
8. Dumitrescu A M, Liao X H, Best T B, Brockmann K and Refetoff S. 2004 A Novel Syndrome Combining Thyroid and Neurological Abnormalities Is Associated with Mutations in a Monocarboxylate Transporter Gene. Am J Hum Genet 74:168-75
9. Schwartz C E, May M M, Carpenter N J, Rogers R C, Martin J, Bialer M G, Ward J, Sanabria J, Marsa S, Lewis J A, Echeverri R, Lubs H A, Voeller K, Simensen R J and Stevenson R E. 2005 Allan-Herndon-Dudley syndrome and the monocarboxylate transporter 8 (MCT8) gene. Am J Hum Genet 77:41-53
10. Allan W, Herndon C N, Dudley F C 1944 Some examples of the inheritance of mental deficiency: apparently sex-linked idiocy and microcephaly. Am J Ment Defic 46:325-334
11. Groeneweg S, van Geest F S, Abaci A, Alcantud A, Ambegaonkar G P, Armour C M, Bakhtiani P, Barca D, Bertini E S, van Beynum I M et al. 2020 Disease characteristics of MCT8 deficiency: an international, retrospective, multicentre cohort study. Lancet Diabetes Endocrinol 8:594-605
12. Dumitrescu A M, Liao X H, Weiss R E, Millen K and Refetoff S. 2006 Tissue-specific thyroid hormone deprivation and excess in monocarboxylate transporter (Mct) 8-deficient mice. Endocrinology 147:4036-4043
13. Trajkovic M, Visser T J, Mittag J, Horn S, Lukas J, Darras V M, Raivich G, Bauer K and Heuer H. 2007 Abnormal thyroid hormone metabolism in mice lacking the monocarboxylate transporter 8. J Clin Invest 117:627-635
14. Mayerl S, Visser T J, Darras V M, Horn S and Heuer H. 2012 Impact of Oatp1c1 deficiency on thyroid hormone metabolism and action in the mouse brain. Endocrinology 153:1528-1537
15. Roberts L M, Woodford K, Zhou M, Black D S, Haggerty J E, Tate E H, Grindstaff K K, Mengesha W, Raman C and Zerangue N. 2008 Expression of the thyroid hormone transporters monocarboxylate transporter-8 (SLC16A2) and organic ion transporter-14 (SLCO1C1) at the blood-brain barrier. Endocrinology 149:6251-6261
16. Vatine G D, Al-Ahmad A, Barriga B K, Svendsen S, Salim A, Garcia L, Garcia V J, Ho R, Yucer N, Qian T, Lim R G, Wu J, Thompson L M, Spivia W R, Chen Z, Van Eyk J, Palecek S P, Refetoff S, Shusta E V and Svendsen C N. 2017 Modeling Psychomotor Retardation using iPSCs from MCT8-Deficient Patients Indicates a Prominent Role for the Blood-Brain Barrier. Cell Stem Cell 20:831-843 e5
17. Mayerl S, Müller J, Bauer R, Richert S, Kassmann C M, Darras V M, Buder K, Boelen A, Visser T J and Heuer H. 2014 Transporters MCT8 and OATP1C1 maintain murine brain thyroid hormone homeostasis. J Clin Invest 124:1987-1999
18. Kersseboom S, Horn S, Visser W E, Chen J, Friesema E C, Vaurs-Barriere C, Peeters R P, Heuer H, Visser T J. 2015 In vitro and mouse studies support therapeutic utility of triiodothyroacetic acid in MCT8 deficiency. Mol Endocrinol 28:1961-1970
19. Horn S, Kersseboom S, Mayerl S, Müller J, Groba C, Trajkovic-Arsic M, Ackermann T, Visser T J and Heuer H. 2013 Tetrac can replace thyroid hormone during brain development in mouse mutants deficient in the thyroid hormone transporter Mct8. Endocrinology 154:968-979
20. Vatine G D, Barrile R, Workman M J, Sances S, Barriga B K, Rahnama M, Barthakur S, Kasendra M, Lucchesi C, Kerns J, Wen N, Spivia W R, Chen Z, Van Eyk J and Svendsen C N. 2019 Human iPSC-Derived Blood-Brain Barrier Chips Enable Disease Modeling and Personalized Medicine Applications. Cell Stem Cell 24:995-1005 e6
21. Vatine G D, Shelest O, Barriga B K, Ofan R, Rabinski T, Mattis V B, Heuer H, Svendsen C N. 2021 Oligodendrocyte progenitor cell maturation is dependent on dual function of MCT8 in the transport of thyroid hormone across brain barriers and the plasma membrane. Glia 69: 2146-2159
22. Mendell J R, Al-Zaidy S, Shell R, Arnold W D, Rodino-Klapac L R, Prior T W, Lowes L, Alfano L, Berry K, Church K, Kissel J T, Nagendran S, L′Italien J, Sproule D M, Wells C, Cardenas J A, Heitzer M D, Kaspar A, Corcoran S, Braun L, Likhite S, Miranda C, Meyer K, Foust K D, Burghes A H M and Kaspar B K. 2017 Single-Dose Gene-Replacement Therapy for Spinal Muscular Atrophy. N Engl J Med 377:1713-1722
23. Mendell J R, Al-Zaidy S A, Lehman K J, McColly M, Lowes L P, Alfano L N, Reash N F, Iammarino M A, Church K R, Kleyn A, Meriggioli M N and Shell R. 2021 Five-Year Extension Results of the Phase 1 START Trial of Onasemnogene Abeparvovec in Spinal Muscular Atrophy. JAMA Neurol 78:834-841
24. Corti M, Liberati C, Smith B K, Lawson L A, Tuna I S, Conlon T J, Coleman K E, Islam S, Herzog R W, Fuller D D, Collins S W and Byrne B J. 2017 Safety of Intradiaphragmatic Delivery of Adeno-Associated Virus-Mediated Alpha-Glucosidase (rAAV1-CMVhGAA) Gene Therapy in Children Affected by Pompe Disease. Hum Gene Ther Clin Dev 28: 208-218
25. Mendell J R, Sahenk Z, Lehman K, Nease C, Lowes L P, Miller N F, Iammarino M A, Alfano L N, Nicholl A, Al-Zaidy S, Lewis S, Church K, Shell R, Cripe L H, Potter R A, Griffin D A, Pozsgai E, Dugar A, Hogan M, Rodino-Klapac L R. 2020 Assessment of Systemic Delivery of rAAVrh74.MHCK7.micro-dystrophin in Children with Duchenne Muscular Dystrophy: A Nonrandomized Controlled Trial. JAMA Neurol 77:1122-1131
26. Eichler F, Duncan C, Musolino P L, Orchard P J, De Oliveira S, Thrasher A J, Armant M, Dansereau C, Lund T C, Miller W P, Raymond G V, Sankar R, Shah A J, Sevin C, Gaspar H B, Gissen P, Amartino H, Bratkovic D, Smith N J C, Paker A M, Shamir E, O'Meara T, Davidson D, Aubourg P and Williams D A. 2017 Hematopoietic Stem-Cell Gene Therapy for Cerebral Adrenoleukodystrophy. N Engl J Med 377:1630-1638
27. Foust K D, Nurre E, Montgomery C L, Hernandez A, Chan C M and Kaspar B K. 2009 Intravascular AAV9 preferentially targets neonatal neurons and adult astrocytes. Nat Biotechnol 27:59-65
28. Iwayama H, Liao X H, Braun L, Barez-Lopez S, Kaspar B, Weiss R E, Dumitrescu A M, Guadalio-Ferraz A and Refetoff S 2016 Adeno Associated Virus 9-Based Gene Therapy Delivers a Functional Monocarboxylate Transporter 8, Improving Thyroid Hormone Availability to the Brain of Mct8-Deficient Mice. Thyroid. 26:1311-1319
29. Mayerl S, Müller J, Bauer R, Richert S, Kassmann C M, Darras V M, Buder K, Boelen A, Visser T J and Heuer H. 2014 Transporters MCT8 and OATP1C1 maintain murine brain thyroid hormone homeostasis. J Clin Invest 124:1987-1999
30. Das M M, Godoy M, Chen S, Moser V A, Avalos P, Roxas K M, Dang I, Yaliez A, Zhang W, Bresee C, Arditi M, Liu G Y, Svendsen C N and Goodridge H S. 2019 Young bone marrow transplantation preserves learning and memory in old mice. Commun Biol 2:3
31. Barez-Lopez S, Grijota-Martinez C, Liao X H, Liao X H, Refetoff S, Guadalio-Ferraz A. 2019 Intracerebroventricular administration of the thyroid hormone analog TRIAC increases its brain content in the absence of MCT8. PLoS One 14:e0226017
32. Sabbagh M F, Heng J S, Luo C, Castanon R G, Nery J R, Rattner A, Goff L A, Ecker J R and Nathans J. 2018 Transcriptional and epigenomic landscapes of CNS and non-CNS vascular endothelial cells. Elife 7:e36187
33. Di Cosmo C, Liao X H, Ye H, Ferrara A M, Weiss R E, Refetoff S and Dumitrescu A M. 2013 Mct8-deficient mice have increased energy expenditure and reduced fat mass that is abrogated by normalization of serum T3 levels. Endocrinology 154:4885-4895
34. van Geest F S, Groeneweg S, Visser W E 2021 Monocarboxylate transporter 8 deficiency: update on clinical characteristics and treatment. Endocrine 71:689-695
35. Bárez-López S, Grijota-Martinez C, Ausó E, Fernández-de Frutos M, Montero-Pedrazuela A and Guadaño-Ferraz A. 2019 Adult Mice Lacking Mct8 and Dio2 Proteins Present Alterations in Peripheral Thyroid Hormone Levels and Severe Brain and Motor Skill Impairments. Thyroid 29:1669-1682
36. López-Espíndola D, Morales-Bastos C, Grijota-Martinez C, Liao X H, Lev D, Sugo E, Verge C F, Refetoff S, Bernal J and Guadaño-Ferraz A. 2014 Mutations of the thyroid hormone transporter MCT8 cause prenatal brain damage and persistent hypomyelination. J Clin Endocrinol Metab 99:E2799-E2804
37. Anbalagan S, Gordon L, Blechman J, Matsuoka R L, Rajamannar P, Wircer E, Biran J, Reuveny A, Leshkowitz D, Stainier D Y R, Levkowitz G. 2018 Pituicyte Cues Regulate the Development of Permeable Neuro-Vascular Interfaces. Dev Cell 47:711-726 e5
38. Ben-Zvi A, Liebner S 2021 Developmental regulation of barrier- and non-barrier blood vessels in the CNS. J Intern Med https://doi.org/10.1111/joim.13263.
39. Ceballos A, Belinchon M M, Sanchez-Mendoza E, Grijota-Martinez C, Dumitrescu A M, Refetoff S, Morte B and Bernal J. 2009 Importance of monocarboxylate transporter 8 for the blood-brain barrier-dependent availability of 3,5,3′-triiodo-L-thyronine. Endocrinology 150:2491-2496
40. Voss J L, Gonsalves B D, Federmeier K D, Tranel D and Cohen N J. 2011 Hippocampal brain-network coordination during volitional exploratory behavior enhances learning. Nat Neurosci 14: 115-120
41. Trajkovic-Arsic M, Visser T J, Darras V M, Friesema E C, Schlott B, Mittag J, Bauer K and Heuer H. 2010 Consequences of monocarboxylate transporter 8 deficiency for renal transport and metabolism of thyroid hormones in mice. Endocrinology 151:802-9
42. Di Cosmo C, Liao X H, Dumitrescu A M, Philp N J, Weiss R E, Refetoff S. 2010 Mice deficient in MCT8 reveal a mechanism regulating thyroid hormone secretion. J Clin Invest 120: 3377-88
43. Fliers E, Unmehopa U A, Alkemade A 2006 Functional neuroanatomy of thyroid hormone feedback in the human hypothalamus and pituitary gland. Mol Cell Endocrinol. 151:1-8
44. Di Cosmo C, Liao X H, Dumitrescu A M, Weiss R E and Refetoff S. 2009 A thyroid hormone analog with reduced dependence on the monocarboxylate transporter 8 for tissue transport. Endocrinology 150:4450-4458
45. Ferrara A M, Liao X H, Ye H, Weiss R E, Dumitrescu A M and Refetoff S. 2015 The thyroid hormone analog DITPA ameliorates metabolic parameters of male mice with Mct8 deficiency. Endocrinology 156:3889-3894
46. Barez-López S, Hartley M D, Grijota-Martínez C, Scanlan T S and Guadaño-Ferraz A. 2018 Sobetirome and its Amide Prodrug Sob-AM2 Exert Thyromimetic Actions in Mct8-Deficient Brain. Thyroid 28:1211-1220
47. Verge C F, Konrad D, Cohen M, Di Cosmo C, Dumitrescu A M, Marcinkowski T, Hameed S, Hamilton J, Weiss R E, Refetoff S. 2012 Diiodothyropropionic acid (DITPA) in the treatment of MCT8 deficiency. J Clin Endocrinol Metab 97:4515-4523
48. Groeneweg S, Peeters R P, Moran C, et al. 2019 Effectiveness and safety of the triiodothyronine analogue Triac in children and adults with MCT8 deficiency: an international, single-arm, open-label, phase 2 trial. Lancet Diabetes Endocrinol 7:695-706
49. Braun D, Schweizer U 2017 The chemical chaperone phenylbutyrate rescues MCT8 mutations associated with milder phenotypes in patients with Allan-Herndon-Dudley syndrome. Endocrinology 158:678-691
50. Braun D, Schweizer U 2015 Efficient activation of pathogenic Aphe501 mutation in monocarboxylate transporter 8 by chemical and pharmacological chaperones. Endocrinology 156:4720-30
51. Mendell J R, Al-Zaidy S, Shell R, et al. 2017 Single-dose gene-replacement therapy for spinal muscular atrophy. N Engl J Med 377:1713-1722
52. White K A, Nelvagal H R, Poole T A, Lu B, Johnson T B, Davis S, Pratt M A, Brudvig J, Assis A B, Likhite S, Meyer K, Kaspar B K, Cooper J D, Wang S and Weimer J M. 2021 Intracranial delivery of AAV9 gene therapy partially prevents retinal degeneration and visual deficits in CLN6-Batten disease mice. Mol Ther—Methods Clin Dev 20:497-507.
53. Pearson T S, Gupta N, San Sebastian W, et al. 2021 Gene therapy for aromatic Lamino acid decarboxylase deficiency by M R-guided direct delivery of AAV2-AADC to midbrain dopaminergic neurons. Nat Commun 12:4251.

### II. DITPA Administration

The Applicant has discovered dosing timings of 3,5-diiodothyropropionic acid (DITPA) that are surprisingly effective for the treatment of Allan-Herndon-Dudley Syndrome (ARDS).

In one embodiment, the present technology is directed to methods of treating ARDS comprising administering DITPA orally twice daily to a pregnant mother who elects to retain an affected male embryo, or for a subject with MCT8 deficiency in need thereof to reduce and normalize high blood T3 ameliorate the hypermetabolism and nutrition given orally three times daily. Wherein administration to a pregnant woman begins no later than 11 weeks after conception of the subject, but preferably between 8 and 10 weeks after conception. The dose of DITPA for pregnant women to treat their affected male fetuses is 0.5±0.2 mg per day and postpartum for affected infants and children of any age, 1.5±0.5 mg per day.

In another embodiment, the present technology is directed to methods of treating Allan-Herndon-Dudley syndrome comprising the following steps:
a) administering DITPA daily at a first dosage for two weeks to a subject in need thereof;
b) administering DITPA daily at a second dosage for two weeks to the subject wherein the second dosage is greater than the first dosage;
c) measuring triiodothyronine (“T3”) serum levels in the subject, wherein if T3 serum levels are normal the second dosage is administered daily;
d) optionally, adjusting daily dosage of DITPA administered to the subject based on T3 serum levels of the subject measured in step c) wherein if the T3 serum levels are too high a third dosage is administered daily wherein the third dosage in greater than the second dosage and wherein if the T3 serum levels are too low a fourth dosage is administered daily wherein the fourth dosage is less than the second dosage; and
e) optionally, measuring T3 serum levels of the subject about 28 days following initial administration of the third or fourth dosage wherein if T3 serum levels are normal the third or fourth dosage is administered daily; and
f) optionally, adjusting daily dosage of DITPA administered to the subject based on T3 serum levels of the subject measured in step e) wherein if the T3 serum levels are too low following daily administration of the third dosage then the subject is administered the second dosage and wherein if the T3 serum levels are too low following daily administration of the fourth dosage then the subject is administered the first dosage daily and wherein if the T3 serum levels are too high following daily administration of the fourth dosage then the subject is administered the second dosage daily.

In a preferred embodiment, the first dosage is about 1 milligram per kilogram of body weight of the subject per day (“mg/kg/day”).

In another preferred embodiment, the second dosage is about 2 mg/kg/day.

In another preferred embodiment, the third dosage is about 2.5 mg/kg/day.

In another preferred embodiment, the fourth dosage is about 1.5 mg/kg/day.

As used herein the term “too high” refers to a T3 serum level that is more than about 15% over T3 serum levels considered normal for the age of the subject.

As used herein the term “too low” refers to a T3 serum level that is more than about 15% under T3 serum levels considered normal for the age of the subject.

As used herein “normal” T3 serum levels by age of the subject is based on levels disclosed in Lem et al., Serum thyroid hormone levels in healthy children from birth to adulthood and in short children born small for gestational age, J Clin Endocrinol Metab, 2012 September, 97(9), 3170-8, doi: 10.1210/jc.2012-1759, Epub 2012 Jun. 26.

In another embodiment, the present disclosure is directed to methods of treating ARDS comprising the following steps:
a) administering DITPA daily at a first dosage for two weeks to a subject in need thereof; and
b) administering DITPA daily at a second dosage for two weeks to the subject wherein the second dosage is greater than the first dosage, wherein daily administration begins three days following birth of the subject.

In a preferred embodiment, the daily dosage of DITPA is administered to a subject in need thereof once a day, more preferably the daily dosage of DITPA is divided in two parts and each part is administered every 12 hours and most preferably the daily dosage of DITPA is divided into three parts and each part is administered every 8 hours.

In a preferred embodiment, administration of DITPA occurs via the oral route.

In one embodiment, DITPA may be formulated in a composition comprising DITPA, or a salt thereof, and one or more pharmaceutically acceptable excipients.

In a preferred embodiment, DITPA, or a salt thereof, may present in the pharmaceutical compositions of the present subject matter at a concentration from about 0.001% to about 10% w/w or w/v.

In a preferred embodiment, the one or more pharmaceutically acceptable excipients may be present in the pharmaceutical compositions of the present disclosure at a concentration from about 90% to about 99.999% w/w or w/v.

Pharmaceutically acceptable excipients suitable for use in the present subject matter include, but are not limited to, disintegrants, binders, fillers, plasticizers, lubricants, permeation enhancers, surfactants, sweeteners, sweetness enhancers, flavoring agents and pH adjusting agents.

The term “disintegrants” as used herein refers to pharmaceutically acceptable excipients that facilitate the disintegration of the tablet once the tablet contacts water or other liquids. Disintegrants suitable for use in the present technology include, but are not limited to, natural starches, such as maize starch, potato starch etc., directly compressible starches such as starch 1500, modified starches such as carboxymethyl starches, sodium hydroxymethyl starches and sodium starch glycolate and starch derivatives such as amylose, cross-linked polyvinylpyrrolidones such as crospovidones, modified celluloses such as cross-linked sodium carboxymethyl celluloses, sodium hydroxymethyl cellulose, calcium hydroxymethyl cellulose, croscarmellose sodium, low-substituted hydroxypropyl cellulose, alginic acid, sodium alginate, microcrystalline cellulose, methacrylic acid-divinylbenzene copolymer salts and combinations thereof.

Binders suitable for use in the present technology include, but are not limited to, polyethylene glycols, soluble hydroxyalkyl celluloses, polyvinylpyrrolidone, gelatins, natural gums and combinations thereof.

Fillers suitable for use in the present technology include, but are not limited to, dibasic calcium phosphate, calcium phosphate tribasic, calcium hydrogen phosphate anhydrous, calcium sulfate and dicalcium sulfate, lactose, sucrose, amylose, dextrose, mannitol, inositol and combinations thereof.

Plasticizers suitable for use in the present subject matter include, but are not limited to, microcrystalline cellulose, triethyl citrate, poly-hexanediol, acetylated monoglyceride, glyceryl triacetate, castor oil, and combinations thereof.

Lubricants suitable for use in the present technology include, but are not limited to, magnesium stearate, sodium stearyl fumarate, stearic acid, glyceryl behenate, micronized polyoxyethylene glycol, talc, silica colloidal anhydrous and combinations thereof.

Permeation enhancers suitable for use in the present subject matter include, but are not limited to, precipitated silicas, maltodextrins, β-cyclodextrins menthol, limonene, carvone, methyl chitosan, polysorbates, sodium lauryl sulfate, glyceryl oleate, caproic acid, enanthic acid, pelargonic acid, capric acid, undecylenic acid, lauric acid, myristic acid, palmitic acid, oleic acid, stearic acid, linolenic acid, arachidonic acid, benzethonium chloride, benzethonium bromide, benzalkonium chloride, cetylpyridium chloride, edetate disodium dihydrate, sodium desoxycholate, sodium deoxyglycolate, sodium glycocholate, sodium caprate, sodium taurocholate, sodium hydroxybenzoyal amino caprylate, dodecyl dimethyl aminopropionate, L-lysine, glycerol oleate, glyceryl monostearate, citric acid, peppermint oil and combinations thereof.

Surfactants suitable for use in the present subject matter include, but are not limited to, sorbitan esters, docusate sodium, sodium lauryl sulphate, cetriride and combinations thereof.

Sweeteners suitable for use in the present technology include, but are not limited to, aspartame, saccharine, potassium acesulfame, sodium saccharinate, neohesperidin dihydrochalcone, sucralose, sucrose, dextrose, mannitol, glycerin, xylitol and combinations thereof.

Sweetness enhancers suitable for use in the present technology include, but are not limited to, ammonium salt forms of crude and refined glycyrrhizic acid.

Flavoring agents suitable for use in the present subject matter include, but are not limited to, peppermint oil, menthol, spearmint oil, citrus oil, cinnamon oil, strawberry flavor, cherry flavor, raspberry flavor, orange oil, tutti frutti flavor and combinations thereof.

pH adjusting agents suitable for use in the present formulation include, but are not limited to, hydrochloric acid, citric acid, fumaric acid, lactic acid, sodium hydroxide, sodium citrate, sodium bicarbonate, sodium carbonate, ammonium carbonate, sodium acetate and combinations thereof.

In another preferred embodiment, the pharmaceutical compositions of the present technology do not contain a preservative.

Pharmaceutical compositions of the present technology may be formulated in any dosage form including but not limited to aerosol including metered, powder and spray, chewable bar, bead, capsule including coated, film coated, gel coated, liquid filled and coated pellets, cellular sheet, chewable gel, concentrate, elixir, emulsion, film including soluble, film for solution and film for suspension, gel including metered gel, globule, granule including granule for solution, granule for suspension, chewing gum, inhalant, injectable including foam, liposomal, emulsion, lipid complex, powder, lyophilized powder and liposomal suspension, liquid, lozenge, ointment, patch, electrically controlled patch, pellet, implantable pellet, pill, powder, powder, metered powder, solution, metered solution, solution concentrate, gel forming solution/solution drops, spray, metered spray, suspension, suspension, syrup, tablet, chewable tablet, coated tablet, coated particles in a tablet, film coated tablet, tablet for solution, tablet for suspension, orally disintegrating tablet, soluble tablet, sugar coated tablet, dispersible tablet, tablet with sensor, tape, troche and wafer and extended release and delayed release forms thereof.

In a preferred embodiment, the pharmaceutical compositions of the present technology are in tablet form. In a more preferred embodiment, the pharmaceutical compositions of the present formulation are in a dispersible tablet form. In an even more preferred embodiment, the pharmaceutical compositions of the present formulation are in a water-dispersible tablet form. In a most preferred embodiment, the pharmaceutical compositions of the present formulation are in a water-dispersible tablet form wherein the tablet is scored such that the tablet is dividable into four equal parts.

In a preferred embodiment, when the pharmaceutical compositions of the present technology are in a water-dispersible tablet form the tablet dispersion time is about 70 seconds or less, more preferably about 60 seconds or less and even more preferably about 40 seconds or less, even more preferably about 30 seconds or less, even more preferably about 20 seconds or less, even more preferably about 10 seconds or less and even more preferably about 5 seconds or less.

As used herein the term “pharmaceutically acceptable” refers to ingredients that are not biologically or otherwise undesirable in an oral application.

As used herein, all numerical values relating to amounts, weights, and the like, are defined as “about” each particular value, that is, plus or minus 10%. For example, the phrase “10% w/w” is to be understood as “9% to 11% w/w.” Therefore, amounts within 10% of the claimed value are encompassed by the scope of the claims.

As used herein “% w/w” refers to the weight percent by weight of the total formulation.

As used herein “% w/v” refers to the weight percent by volume of the total formulation.

As used herein the term “effective amount” refers to the amount necessary to treat a subject in need thereof.

As used herein the term “treatment” or “treating” refers to alleviating or ameliorating ARDS or symptoms of ARDS.

As used herein, the term “stable” includes, but is not limited to, physical and chemical stability.

Pharmaceutically acceptable salts of that can be used in accordance with the current subject matter include but are not limited to hydrochloride, dihydrate hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, mesylate, maleate, gentisinate, fumarate, tannate, sulphate, tosylate, esylate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts.

Throughout the application, the singular forms “a,” “an,” and “the” include plural reference unless the context clearly dictates otherwise.

The disclosed embodiments are simply exemplary embodiments of the inventive concepts disclosed herein and should not be considered as limiting unless the claims expressly state otherwise.

### III. Combining Gene Therapy and DITPA Administration

Administration of DITPA may be used to reduce and normalize high blood T3, ameliorating the hypermetabolism and nutrition but not the neuropsychomotor deficit associated with ARDS. Use of gene therapy helps correct the neurological abnormalities, learning and recall abilities, but not the high blood T3 causing the increased metabolism. A combination of DITPA treatment and gene therapy corrects both neuropsychomotor and metabolic defects that each treatment alone could not achieve, providing full rescue of the genetic defect.

In one embodiment, the present technology is directed to methods of using a combination of DITPA administration to a subject, along with gene therapy, to treat and correct neurological and metabolic abnormalities associated with ARDS in a subject in need of such treatment, wherein the gene therapy comprises administration of AAV9-MCT8 to the subject, and wherein the DITPA is administered daily, to reduce and normalize high blood T3 and ameliorate the hypermetabolism and nutrition in the subject, and preferably where the AAV9-MCT8 is administered intravenously to the subject.

The following examples are intended to illustrate the present technology and to teach one of ordinary skill in the art how to use the formulations of this new technology. They are not intended to be limiting in any way.

### EXAMPLES

### Example 1—Dosing Regimen for a Pre-Natal Subject (Prophetic)

### Method

DITPA was administered to a pregnant mother of a male pre-natal subject that had previously tested positive for the SLC16A2 allele correlated with Allan-Herndon-Dudley syndrome at a daily dosage of 1 mg/kg/day divided over three administration spaced 8 hours apart starting at 4 weeks after conception and ending at birth of the subject.

### Results

The dosing regimen successfully reduced symptoms of ARDS in the newborn subject as compared to affected newborns whose mothers were not treated with DITPA.

### Example 2—In Vitro Evidence of Direct Effect of SRW-101 (DITPA) in Decreasing the T3 Generated from T4: SRW101 (DITPA) Inhibiting D1 Enzymatic Activity in Liver In Vitro

DITPA reduces the activity of deiodinase-1 in vivo and in vitro in liver (see figure shown Dose response of DITPA added to liver in vitro and measurement of D1 enzymatic activity, i.e., conversion of T4 to T3). This is the main mechanism of reduction in T3 and increase in T4 by reducing its consumption. It was shown to occur in humans with MCT8 deficiency.

These are the consequences of the normalization of serum T3 levels, a critical endocrine biomarker and parameter for therapeutic efficacy, as they measure the anticipated metabolic changes resulting from the normalization of the thyroid tests.

More specifically, the reduction of T3, which acts on peripheral tissue to accelerate the metabolism, is expected to improve nutrition and increase the ability to gain weight.

Important measurements such as weight gain (corrected for age) and metabolic parameters (cholesterol, creatine kinase, SHBG) are secondary endpoints.

Annotated observations by the parents such as sleep, food record, motor activity are of immense value.

Dose response of DITPA added to liver in vitro and measurement of D1 enzymatic activity (conversion of T4 to T3)

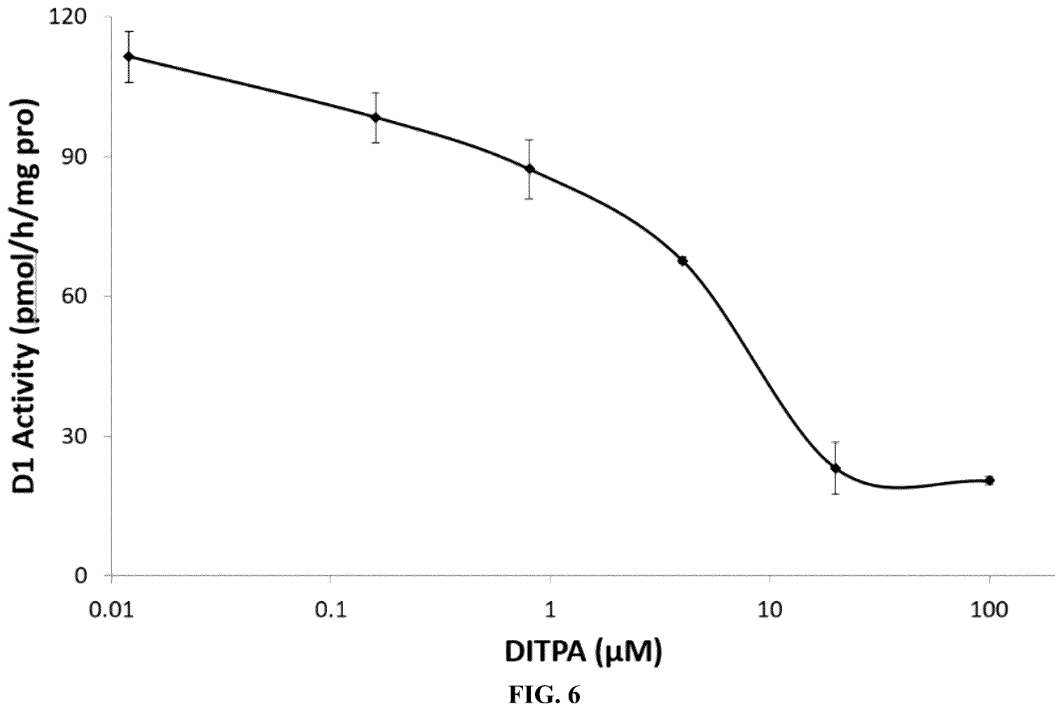

FIG. 6 demonstrates in vitro evidence of direct effect of DITPA in decreasing the T3 generated from T4, rather than reducing it through decrease in T4 by TSH suppression, as is the case with TRIAC. T4 is important to the brain even in the presence of reduced uptake due to MCT8 deficiency. The data in FIG. 6 reflects the effect of adding DITPA to liver (in vitro) as measured by D1 enzymatic activity, namely conversion of T4 to T3.

### Example 3—Background Understanding of Thyroid Physiology

Research centered on regulation of gene expression and gene therapy, with three aims.
(1) Determine the mechanism by which several newly identified genetic defects produce the observed thyroid phenotypes. These include the selenoenzyme deiodinases D1 and D3; PKHD1L1 mutations by studying the Pkdhl11KO mice; and LRP2 mutations by in vitro structural and functional characterization.
(2) Determine the mechanism of resistance to TSH (RTSH) caused by mutations in a primate-specific short tandem repeat (STR) on chromosome-15. Human thyroid organoids recently developed in collaboration will be used to generate STR mutant thyroid organoids using CRISPR/Cas9 or PiggyBac transposon as a genome editing tool, in order to study the physiological function of this primate specific STR and its role in the dominantly inherited phenotype of RTSH. TSH sensitivity of normal and mutant organoids will be determined in vitro or in vivo after transplantation into hypothyroid mice.
(3) Determine the effectiveness of combined gene and thyroid hormone (TH) analogue treatments in monocarboxylate 8 (MCT8) deficiency. The X chromosome linked MCT8 deficiency produces in boys a disease known as Allan-Herndon-Dudley-Syndrome (ARDS) with severe neuropsychomotor defects, caused by deficiency of TH transport in brain, and systemic thyrotoxicosis caused by excess of circulating T₃. Double knockout (dKO) mice, lacking Mct8 and the TH transporter Oatp1c1, recapitulate the findings of AHDS.

We recently showed that gene therapy in peripubertal dKO mice with adeno associated virus 9 (AAV9) containing the human MCT8 cDNA improved the locomotor and cognitive function by near normalization of brain T3 content but failed to correct the serum thyroid tests. Adding the TH analogues diiodothyropropionic acid (DITPA) or triiodothyroacetic acid (TRIAC), known to correct the thyrotoxicosis of ARDS in peripheral tissues but not the neuropsychomotor manifestations, is intended to achieve rescue of this incapacitating disease.

### The Specific Aims

### Aim #3. Determine the Effectiveness of Combined Gene Therapy and TH Analogue Treatments in Mct8 Deficiency.

Rationale & Impact: Deficiency of the X-linked MCT8 results in a complex phenotype in boys that includes a severe neuropsychomotor defect, caused by deficiency of TH transport in the brain, and systemic thyrotoxicosis caused by excess of circulating T3. Treatment with the TH analogues diiodothyropropionic acid (DITPA) or triiodothyroacetic acid (TRIAC) corrects the thyrotoxicosis of peripheral tissues but not the neuropsychomotor defect. Double knockout (dKO) mice, lacking Mct8 and the TH transporter Oatp1c1, recapitulate the findings of ARDS. We recently showed that gene therapy in peripubertal dKO mice with adeno associated virus 9 (AAV9), containing the human MCT8 cDNA, improved the locomotor and cognitive function by near normalization of brain T3 content but failed to correct the serum TFTs. Combined treatment with AAV9 and TH analogues has the potential to rescue MCT8 deficiency and serve as a preclinical model for treatment in humans.

We Will:
1. Treat dKO mice with AAV9-hMCT8 alone and together with the TH analogues DITPA and TRIAC. Serum TFT and tissue content of T3, T4 and analogues as well as the expression of TH responsive genes will be assessed in different brain areas and organs.

In addition to locomotor function, the metabolic and cardiac effects will be assesses using metabolic cages.

### Research Strategy

### Significance

We have discovered defects in the TH transmembrane transporter monocarboxylate 8 (MCT8)⁶ causing the severe neuro-psychomotor syndrome [Allan-Herndon-Dudley syndrome (AHDS)] and defects in TH metabolism, SBP2⁷ and DIO1⁸.

We investigate highly significant areas, namely (i) expand the study of newly identified genetic defects that affect thyroid physiology including the deiodinases (ii) develop a potentially effective treatment for MIDS. Preliminary data make this proposal both clinically and scientifically relevant, emphasizing its translational strength.

Another significant aspect is the development of a treatment for AHDS. Those who have seen a child affected by AIMS will readily understand the urgent need for an effective treatment. While the current focus is on the use of TH analogues that correct only the hypermetabolism, we recently developed a promising gene therapy strategy. Our compelling new data show that using adeno-associated virus 9 (AAV9) expressing human MCT8 cDNA ameliorates the neuro-psychomotor component of ARDS even when administered in peripubertal life¹⁵. Combined with TH analogues, gene therapy holds great promise for long-term improvement of the global phenotype of ARDS.

### Innovation

Treatment of patients with ARDS' has remained an important challenge in thyroidology. We propose a pioneering approach for the thyroid field using AAV9 transduction of normal human MCT8 cDNA to deliver functional hMCT8 at juvenile stage to a mouse model of ARDS (mice deficient in Mct8 and Oatp1c1) which partially rescues the neuromotor abnormality¹⁵, with concomitant use of TH analogue treatments to correct the hypermetabolism^{18,19}. This combined approach targets the complex pathophysiology of the MCT8 deficiency with the potential to rescue or attenuate the severe phenotype of this defect and serve as a preclinical model for treatment in humans.

### Aim 3. Determine the Effectiveness of Combined Gene Therapy and TH Analogue Treatments in MCT8 Deficiency.

Since the discovery of the MCT8 deficiency^{6,24} resulting in the severe neurological manifestations due to impaired TH transport in the brain, and thyrotoxicosis in other tissues, multiple patients have been identified. However, treatment remains challenging. The use of TH analogues improved the hypermetabolic state but failed to improve the neuropsychomotor manifestations. We showed that intravenous (IV) administration of adeno-associated virus serotype 9 vector expressing the human MCT8 cDNA (AAV9-hMCT8) to 1-day old Mct8KO mice, populated their choroid plexus with the hMCT8 protein. Their brain T3 content increased, as well as expression of genes regulated by TH²⁵. When injected to 30-day old Mct8 and Oatp1c1 deficient (dKO) mice, manifesting thyroid and neurological abnormalities, improvement was observed in locomotor performance, learning and recall ability¹⁵. This was accompanied by an increase in brain T3 content and TH-regulated gene expression¹⁵. However, little to no changes were observed in serum and peripheral tissues. We now propose a combination of gene therapy together with TH analogues to improve both hypermetabolism and the neuropsychomotor deficitt²⁶.

### Example 5—Gene Therapy

### Specific Aim #3. Determine the Effectiveness of Combined Gene and TH Analogue Treatments in Mct8 Deficiency.

Background: MCT8 deficiency manifests a syndrome with two components: severe neurodevelopmental delay with gait disturbance, dystonia, poor head control and mental retardation as well as a characteristic pattern of thyroid tests abnormalities including increased serum T₃ and TSH, and decreased T₄ and rT₃^{6,24}. Previously described in 1944 by Allan, Herndon and Dudley⁶⁰ as X-linked mental retardation in males, and now referred to as AHDS, the syndrome was subsequently found to be caused by mutations in the MCT8 gene⁶¹.

Mouse models of Mct8 deficiency replicate the thyroid tests abnormalities in humans^{62,63}. However, they do not manifest neurological abnormalities owing to the expression in brain of Oatp1c1, another TH cell membrane transporter, present in very small amounts in human brain⁶⁴. Indeed, a mouse deficient in both Mct8 and Oatp1, or double KO (dKO), manifests both thyroid and neuromotor defects⁶⁵.

It is now clear that a selective brain TH deficiency is produced by severe reduction in hormone transport at the level of vascular endothelial cells or the blood brain barrier (BBB)^{66,67}. Thus, MCT8 deficiency produces TH deficiency in brain leading to the neuromotor abnormalities, and TH excess from increased circulating T3 that reaches tissues by alternative transporters, causes cardiotoxicity and hypermetabolism. Thus, contrary to TH deficiency caused by congenital absence of a thyroid gland or due to a defect in TH synthesis, the combined selective tissue deficiency and excess of MCT8 deficiency cannot be treated with TH replacement⁶⁸.

Devising a new treatment that influences both components of the defect is proposed in this aim. This will require a combination of treatments to improve both hypermetabolism and the neuro psychomotor deficit, with the goal of altering the natural history of the defect²⁶ through improved mobility, reduction in complications, increased survival, and facilitating long term care.

Rationale: Studies in Mct8 deficient mice showed that the TH analogues diiodothyropropionic acid (DITPA) or triiodothyroacetic acid (TRIAC) enter cells independent of MCT8^{69,70}. Trials using these analogues in children with MCT8 deficiency reduce the high serum T3 levels and diminish the hypermetabolism, the heart rate and improve some parameters that reflect TH action on peripheral tissues. However, they produced no significant effect on the neuropsychomotor deficit^{19,71} with no documented change in neurodevelopment even with prolonged TRIAC use¹⁸.

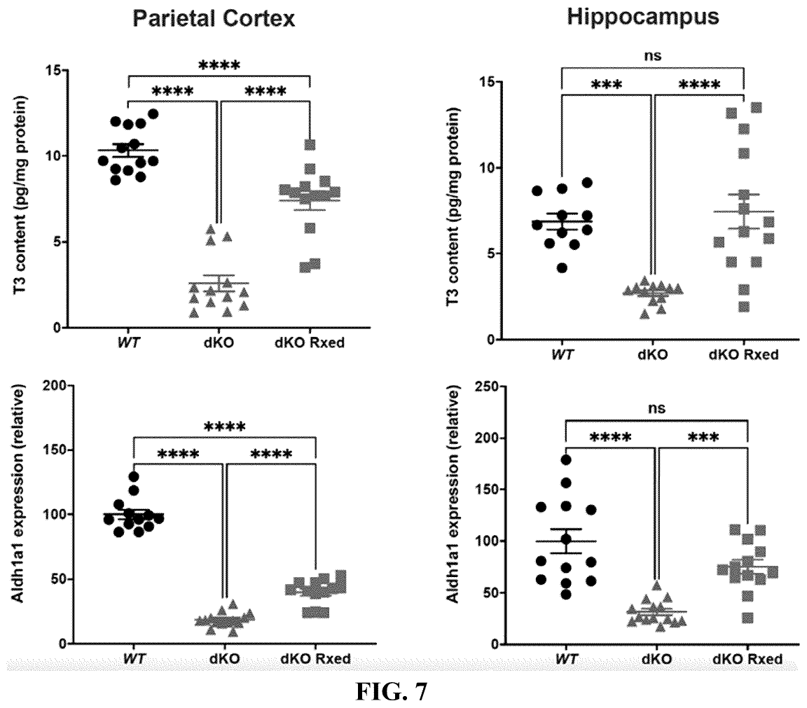

We resorted to gene therapy using an adeno-associated virus serotype 9 (AAV9) vector shown to rescue spinal muscular atrophy⁷² and that is FDA approved. We showed that AAV9 carrying human MCT8 cDNA, when given IV to 1-day old Mct8 deficient mice, increased brain T3 content and expression of genes positively regulated by TH, through populating the choroid plexus with the hMCT8 protein²⁵. When injected to 30-day old dKO mice that manifest thyroid and neurological abnormalities, improvement was observed in locomotor performance, learning and recall ability¹⁵. This was accompanied by an increase in T3 content and TH-regulated gene expression in all three brain areas examined: thalamus, hippocampus and parietal cortex in males (FIG. 7)¹⁵.

However, little to no changes were observed in liver markers despite important expression of hMCT8 in this tissue, nor in serum TH concentration except for a minimal decrease in the T₃/rT₃ ratio¹⁵. These early results of gene therapy are very encouraging as a single treatment has a lasting effect (more than 100 days) on brain function in a mouse model with close resemblance to the AHDS in humans. More importantly, it is still effective when applied at P30. Thus, to further improve the potential gene therapy, we propose to combine it with a treatment that is able to reduce the serum T3 concentration.

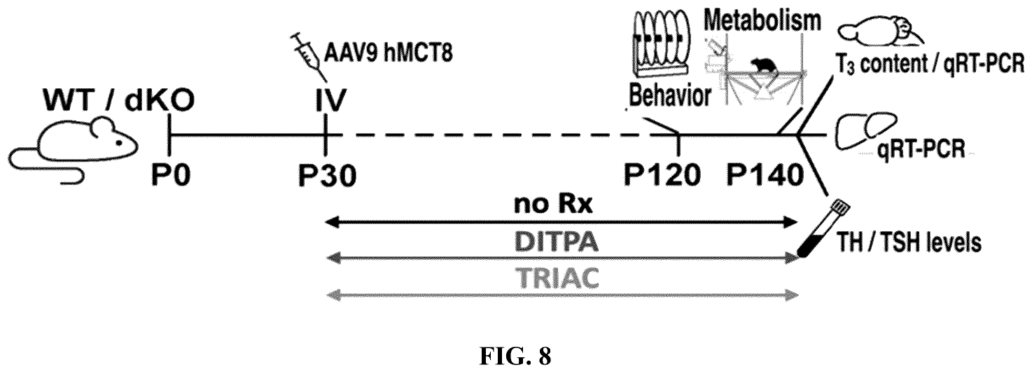

Experimental Approach: The proposed protocol is schematically presented in FIG. 8.

AAV9-hMCT8 containing 50×10¹⁰ viral particles/g will be injected in the tail vein of P30 dKO mice. WT mice will receive the same amount of empty viral vector (AAV9 without hMCT8). Three days later, groups of 10 animals will be treated with doses of DITPA and TRIAC that normalize the serum T₃ concentration of dKO mice. Given by daily intraperitoneal injections, these are per 100 g BW, 0.3 mg DITPA and 6 μg TRIAC. (1) Baseline measurements of serum T₄, T₃, rT₃, TSH, cholesterol, alkaline phosphatase (AP) and creatine kinase (CK) will be obtained a day before. The same tests will be repeated 10 days later (P40) and the doses of DITPA and TRIAC will be adjusted according to the serum levels of T₃, while T₄ will be kept at a normal level of 3.5-5.0 μg/dL for adult C57B1/6J male mice.

Another blood sample will be obtained at P60 and again at P120. (2) Behavioral and locomotor tests (rotarod, open field, gate analysis, Barnes maze, Y maze) will be carried out over a period of 20 days. During the same interval of time, and while not undergoing behavioral and locomotor tests, metabolic studies will be performed using indirect calorimetry in metabolic cages as previously carried out in the laboratory⁷³, to assess the metabolic and cardiac effects of the combination treatment. Total energy expenditure (TEE), Respiratory Exchange Ratio (RER), total activity, food and water intake and heart rate will be measured. Physical activity will be continuously measured, as well as O₂ uptake and CO₂ production at 30 min intervals. RER, TEE, glucose, and lipid oxidation will be calculated from the 02 consumption (VO₂) and CO₂ production (VCO₂) relative to body weight.

On P140, after collection of a terminal blood sample, animals will be perfused under anesthesia, prior to tissue collection. Tissues from two animals from each group will be used for immunohistochemistry and confocal microscopy to localize the hMCT8 protein. The following tissues will be collected and immediately frozen: whole brain, anterior pituitary, thyroid, a fragment of liver, kidney, heart, and muscle (gastrocnemius). Subsequently, the frozen brain will be dissected to recover frontal, parietal and occipital cortices, hippocampus, thalamus, hypothalamus, striatum, choroid plexus, and cerebellum. Tissues will be analyzed for T₃, T₄ and respective analogues (DITPA and TRIAC) content as well as expression of tissue specific TH-regulated genes and enzymatic activities of the three deiodinases.

The expression of the following genes will be measured by qPCR. In brain (Hr, Dio2, Dio3, Aldh1a1, Cbr2); in pituitary (Tshb, Gh, Trhr, Dio2); in thyroid (Tshr, Tg, Tpo, Duox2, Nis, Ttf1); in liver (Dio1, Usp2, Me1); in heart (Serca2, Myh6, Myh7), in muscle (Hr, Myh1, 2 and 7, Mct4, Pkm); Kidney (Dio1, NaPi2, Clc2). The effect on other transporters (Mct10, Lat1, Lat2) and the injected human MCT8 will be also measured. It should be noted that while some monoclonal antibodies can measure T₃ without interference from DITPA⁷⁴, this is not the case with TRIAC⁷⁵. Thus, T₃ together with DITPA and TRIAC, when appropriate, will be measured by liquid cromatography-tandem mass spectrometry (LC-MS/MS)⁴⁵.

### ANTICIPATED RESULTS, POTENTIAL DIFFICULTIES, ADDITIONAL EXPERIMENTS, ALTERNATIVES

Based on this information the following effects are anticipated. (a) DITPA should normalize all three iodothyronines as observed in vivo¹⁹ by decreasing the D1 activity⁷³, an effect demonstrated in vitro (FIG. 6). This will reduce the hypermetabolism as evidenced by changes in markers of TH action (b) TRIAC should also reduce the serum T₃, however, most likely by decreasing serum T₄ through a central mechanism resulting in a decreased TSH⁷¹.

Different from DITPA, TRIAC increases D1⁷⁶ and adding L-T₄ further increases the T₃⁷¹. However, the resulting reduction of serum T₄ which acts as obligatory precursor of T₃ in the brain⁷⁷, may dampen the effect of the gene therapy. As all treatments have been used separately in the past, it is anticipated that gene therapy in combination with both DITPA and TRIAC will produce a combination of the effects observed with the individual treatments as described above. In addition, unbiased approaches such as RNAseq can be used to assess for transcriptional changes in tissues in the setting of combined treatment with AAV9 mediated gene therapy and TH analogue.

### REFERENCES

1. Sakurai A, Takeda K, Ain K, Ceccarelli P, Nakai A, Seino S, Bell G I, Refetoff S, DeGroot L J. Generalized resistance to thyroid hormone associated with a mutation in the ligand-binding domain of the human thyroid hormone receptor b. Proc Natl Acad Sci (USA). 1989; 86:8977-8981.
2. Sunthornthepvarakul T, Gottschalk M E, Hayashi Y, Refetoff S. Resistance to thyrotropin caused by mutations in the thyrotropin-receptor gene. N Engl J Med. 1995; 332:155-160.
3. Mori Y, Seino S, Takeda K, Flink I L, Murata Y, Bell G I, Refetoff S. A mutation causing reduced biological activity and stability of thyroxine-binding globulin probably as a result of abnormal glycosylation of the molecule. Mol Endocrinol. 1989; 3:575-579.
4. Sunthornthepvarakul T, Angkeow P, Weiss R E, Hayashi Y, Refetoff S. A identiucal missense mutation in the albumin gene produces familial disalbuminemic hyperthyroxinemia in 8 unrelated families. Biochem Biophys Res Commun. 1994; 202:781-787.
5. Pohlenz J, Dumitrescu A, Zundel D, Martine U, Schonberger W, Koo E, Weiss R E, Cohen R N, Kimura S, Refetoff S. Partial deficiency of thyroid transcription factor 1 produces predominantly neurological defects in humans and mice. J Clin Invest. 2002; 109:469-473.
6. Dumitrescu A M, Liao X H, Best T B, Brockmann K, Refetoff S. A Novel syndrome combining thyroid and neurological abnormalities Is associated with mutations in a monocarboxylate transporter gene. Am J Hum Genet. 2004; 74(1):168-175.
7. Dumitrescu A M, Liao X-H, Abdullah S Y M, Lado-Abeal J, Abdul-Majed F, Moeller L C, Boran G, Schomburg L, Weiss R E, Refetoff S. Mutations in SECISBP2 result in abnormal thyroid hormone metabolism. Nat Genet. 2005; 37(11):1247-1252.
8. Franca M M, German A, Fernandes G W, Liao X H, Bianco A C, Refetoff S, Dumitrescu A M. Human Type 1 Iodothyronine Deiodinase (DIO1) Mutations Cause Abnormal Thyroid Hormone Metabolism. Thyroid. 2021; 31(2):202-207.
9. Hernandez A, Fiering S, Martinez E, Galton V A, St Germain D. The gene locus encoding iodothyronine deiodinase type 3 (Dio3) is imprinted in the fetus and expresses antisense transcripts. Endocrinology 2002; 143 (11):4483-4486.
10. Charalambous M, Hernandez A. Genomic imprinting of the type 3 thyroid hormone deiodinase gene: regulation and developmental implications. Biochim Biophys Acta. 2013; 1830(7):3946-3955.
11. Kagami M, Kurosawa K, Miyazaki O, Ishino F, Matsuoka K, Ogata T. Comprehensive clinical studies in 34 patients with molecularly defined UPD(14)pat and related conditions (Kagami-Ogata syndrome). Eur J Hum Genet. 2015; 23(11):1488-1498.
12. Kagami M, Nagasaki K, Kosaki R, Horikawa R, Naiki Y, Saitoh S, Tajima T, Yorifuji T, Numakura C, Mizuno S, Nakamura A, Matsubara K, Fukami M, Ogata T. Temple syndrome: comprehensive molecular and clinical findings in 32 Japanese patients. Genet Med. 2017; 19(12):1356-1366.
13. Grasberger H, Mimouni-Bloch A, Vantyghem M-C, van Vliet G, Abramowicz M, Metzger D L, Abdullatif H, Rydlewski C, Macchia P E, Scherberg N H, van Sande J, Mimouni M, Weiss R E, Vassart G, Refetoff S. Autosomal dominant Resistance to thyrotropin as a distinct entity in five multigenerational kindreds: clinical characterization and exclusion of candidate-loci. J Clin Endocrinol Metab. 2005; 90(7):4025-4034.
14. Grasberger H, Vaxillaire M, Pannain S, Beck J C, Mimouni-Bloch A, Vatin V, Vassart G, Froguel P, Refetoff S. Identification of a locus for nongoitrous congenital hypothyroidism on chromosome 15q25.3-26.1. Hum Genet. 2005; 118(3-4):348-355.
15. Liao X H, Avalos P, Shelest O, Ofan R, Shilo M, Bresee C, Likhite S, Vit J P, Heuer H, Kaspar B, Meyer K, Dumitrescu A M, Refetoff S, Svendsen C N, Vatine G D. AAV9-MCT8 delivery at juvenile stage ameliorates neurological and behavioral deficits in a mouse model of MCT8-deficiency. Thyroid. 2022. https://doi.org/10.1089/thy.2022.0034
16. Romitti M, de Faria da Fonsecaa B, G. D, P. G, A. T, E. ES, Van Simaeys G, Chomette L, Lasolle H, Monestier O, Figini Kasprzyk D, Detours V, Pal Singh S, Goldman G, Refetoff S, Costagliola S. Transplantable human thyroid organoids generated from embryonic stem cells to rescue hypothyroidism. 2021. https://doi.org/10.1101/2021.12.01.470729
17. Grijota-Martínez C, Bárez-López S, Gómez-Andrés D, Guadaño-Ferraz A. MCT8 Deficiency: The Road to Therapies for a Rare Disease. Frontiers in Neuroscience. 2020; 14.
18. van Geest F S, Groeneweg S, van den Akker E L T, Bacos I, Barca D, van den Berg SAA, Bertini E, Brunner D, Brunetti-Pierri N, Cappa M, Cappuccio G, Chatterjee K, Chesover A D, Christian P, Coutant R, Craiu D, Crock P, Dewey C, Dica A, Dimitri P, Dubey R, Enderli A, Fairchild J, Gallichan J, Garibaldi L R, George B, Hackenberg A, Heinrich B, Huynh T, Klosowska A, Lawson-Yuen A, Linder-Lucht M, Lyons G, Lora F M, Moran C, Muller K E, Paone L, Paul P G, Polak M, Porta F, Reinauer C, de Rijke Y B, Seckold R, Menevse T S, Simm P, Simon A, Spada M, Stoupa A, Szeifert L, Tonduti D, van Toor H, Turan S, Vanderniet J, de Waart M, van der Wal R, van der Walt A, van Wermeskerken A M, Wierzba J, Zibordi F, Zung A, Peeters R P, Visser W E. Long-term efficacy of T₃ analogue Triac in children and adults with MCT8 deficiency: a real-life retrospective cohort study. J Clin Endocrinol Metab. 2022; 107(3): el136-e1147.
19. Verge C F, Konrad D, Cohen M, Di Cosmo C, Dumitrescu A M, Marcinkowski T, Hameed S, Hamilton J, Weiss R E, Refetoff S. Diiodothyropropionic Acid (DITPA) in the Treatment of MCT8 Deficiency. J Clin Endocrinol Metab. 2012; 97(12):4515-4523.
20. Iwayama H, Kakita H, Iwasa M, Adachi S, Takano K, Kikuchi M, Fujisawa Y, Osaka H, Yamada Y, Okumura A, Hirani K, Weiss R E, Refetoff S. Measurement of Reverse Triiodothyronine Level and the Triiodothyronine to Reverse Triiodothyronine Ratio in Dried Blood Spot Samples at Birth May Facilitate Early Detection of Monocarboxylate Transporter 8 Deficiency. Thyroid 2021; 31(9):1316-1321.
21. Grasberger H, Refetoff S. Resistance to thyrotropin. Best Pract Res Clin Endocrinol Metab. 2017; 31(2): 183-194.
22. Aliesky H, Courtney C L, Rapoport B, McLachlan S M. Thyroid autoantibodies are rare in nonhuman great apes and hypothyroidism cannot be attributed to thyroid autoimmunity. Endocrinology. 2013; 154(12):4896-4907.
23. Antonica F, Kasprzyk D F, Opitz R, Iacovino M, Liao X H, Dumitrescu A M, Refetoff S, Peremans K, Manto M, Kyba M, Costagliola S. Generation of functional thyroid from embryonic stem cells. Nature. 2012; 491(7422):66-71.
24. Friesema E C, Grueters A, Biebermann H, Krude H, von Moers A, Reeser M, Barrett T G, Mancilla E E, Svensson J, Kester M H, Kuiper G G, Balkassmi S, Uitterlinden A G, Koehrle J, Rodien P, Halestrap A P, Visser T J. Association between mutations in a thyroid hormone transporter and severe X-linked psychomotor retardation. Lancet. 2004; 364(9443):1435-1437.
25. Iwayama H, Liao X H, Braun L, Barez-Lopez S, Kaspar B, Weiss R E, Dumitrescu A M, Guadano-Ferraz A, Refetoff S. Adeno Associated Virus 9-Based Gene Therapy Delivers a Functional Monocarboxylate Transporter 8, Improving Thyroid Hormone Availability to the Brain of Mct8-Deficient Mice. Thyroid. 2016; 26(9):1311-1319.
26. Groeneweg S, van Geest F S, Abaci A, Alcantud A, Ambegaonkar G P, Armour C M, Bakhtiani P, Barca D, Bertini E S, van Beynum I M, Brunetti-Pierri N, Bugiani M, Cappa M, Cappuccio G, Castellotti B, Castiglioni C, Chatterjee K, de Coo IFM, Coutant R, Craiu D, Crock P, DeGoede C, Demir K, Dica A, Dimitri P, Dolcetta-Capuzzo A, Dremmen MHG Dubey R, Enderli A, Fairchild J, Gallichan J, George B, Gevers E F, Hackenberg A, Halasz Z, Heinrich B, Huynh T, Klosowska A, van der Knaap M S, van der Knoop Konrad D, Koolen D A, Krude H, Lawson-Yuen A, Lebl J, Linder-Lucht M, Lorea C F, Lourenco C M, Lunsing R J, Lyons G, Malikova J, Mancilla E E, McGowan A, Mericq V, Lora F M, Moran C, Muller K E, Oliver-Petit I, Paone L, Paul P G, Polak M, Porta F, Poswar F O, Reinauer C, Rozenkova K, Menevse T S, Simm P, Simon A, Singh Y, Spada M, van der Spek J, Stals MAM, Stoupa A, Subramanian G M, Tonduti D, Turan S, den Uil C A, Vanderniet J, van der Walt A, Wemeau J L, Wierzba J, de Wit M Y, Wolf N I, Wurm M, Zibordi F, Zung A, Zwaveling-Soonawala N, Visser W E. Disease characteristics of MCT8 deficiency: an international, retrospective, multicentre cohort study. Lancet Diabetes Endocrinol. 2020; 8(7): 594-605.
27. Russo S C, Salas-Lucia F, Bianco A C. Deiodinases and the Metabolic Code for Thyroid Hormone Action. Endocrinology. 2021; 162(8).
28. Dentice M, Marsili A, Zavacki A, Larsen P R, Salvatore D. The deiodinases and the control of intracellular thyroid hormone signaling during cellular differentiation. Biochim Biophys Acta. 2013; 1830(7):3937-3945.
29. Bianco A C, Larsen P R. Cellular and structural biology of the deiodinases. Thyroid. 2005; 15(8):777-786.
30. Schweizer U, Schlicker C, Braun D, Kohrle J, Steegborn C. Crystal structure of mammalian selenocysteine-dependent iodothyronine deiodinase suggests a peroxiredoxin-like catalytic mechanism. Proc Natl Acad Sci USA. 2014; 111(29): 10526-10531.
31. Callebaut I, Curcio-Morelli C, Mornon Gereben B, Buettner C, Huang S, Castro B, Fonseca T L, Harney J W, Larsen P R, Bianco A C. The Iodothyronine Selenodeiodinases Are Thioredoxin-fold Family Proteins Containing a Glycoside Hydrolase Clan GH-A-like Structure*. Journal of Biological Chemistry 2003; 278(38): 36887-36896.
32. Jumper J, Evans R, Pritzel A, Green T, Figurnov M, Ronneberger O, Tunyasuvunakool K, Bates R, Židek A, Potapenko A, Bridgland A, Meyer C, Kohl SAA, Ballard A J, Cowie A, Romera-Paredes B, Nikolov S, Jain R, Adler J, Back T, Petersen S, Reiman D, Clancy E, Zielinski M, Steinegger M, Pacholska M, Berghammer T, Bodenstein S, Silver D, Vinyals Senior A W, Kavukcuoglu K, Kohli P, Hassabis D. Highly accurate protein structure prediction with AlphaFold. Nature. 2021; 596(7873):583-589.
33. Wajner S M, Goemann I M, Bueno A L, Larsen P R, Maia A L. IL-6 promotes nonthyroidal illness syndrome by blocking thyroxine activation while promoting thyroid hormone inactivation in human cells. J Clin Invest. 2011; 121(5):1834-1845.
34. Baqui M M, Gereben B, Harney J W, Larsen P R, Bianco A C. Distinct subcellular localization of transiently expressed types 1 and 2 iodothyronine deiodinases as determined by immunofluorescence confocal microscopy. Endocrinology 2000; 141(11):4309-4312.
35. Baqui M, Botero D, Gereben B, Curcio C, Harney J W, Salvatore D, Sorimachi K, Larsen P R, Bianco A C. Human Type 3 Iodothyronine Selenodeiodinase Is Located in the Plasma Membrane and Undergoes Rapid Internalization to Endosomes*. Journal of Biological Chemistry 2003; 278(2): 1206-1211.
36. Cicatiello A G, Di Girolamo D, Dentice M. Metabolic Effects of the Intracellular Regulation of Thyroid Hormone: Old Players, New Concepts. Front Endocrinol (Lausanne). 2018; 9:474-474.
37. Hernandez A, Martinez E, Fiering S, Parlow A, Galton V, St. Germain D. The type 3 deiodinase (D3)-deficient mouse provides a new model of developmental central hypothyroidism. Paper presented at: 76th Annual Meeting of the American Thyroid Association 2004; Vancouver, BC, Canada.
38. Williams A J, Robson H, Kester MHA, van Leeuwen J P T M, Shalet S M, Visser T J, Williams G R. Iodothyronine deiodinase enzyme activities in bone. Bone. 2008; 43(1):126-134.
39. Ng L, Lyubarsky A, Nikonov S S, Ma M, Srinivas M, Kefas B, St Germain D L, Hernandez A, Pugh E N, Jr., Forrest D. Type 3 deiodinase, a thyroid-hormone-inactivating enzyme, controls survival and maturation of cone photoreceptors. J Neurosci. 2010; 30(9):3347-3357.
40. Kurdyukov S, Bullock M. DNA Methylation Analysis: Choosing the Right Method. Biology (Basel). 2016; 5(1):3.
41. Martinez M E, Hernandez A. The Type 3 Deiodinase Is a Critical Modulator of Thyroid Hormone Sensitivity in the Fetal Brain. Frontiers in Neuroscience. 2021; 15.
42. Mancino G, Sibilio A, Luongo C, Di Cicco E, Miro C, Cicatiello A G, Nappi A, Sagliocchi S, Ambrosio R, De Stefano M A, Di Girolamo D, Porcelli T, Murolo M, Saracino F, Perruolo G, Formisano P, Stornaiuolo M, Dentice M. The Thyroid Hormone Inactivator Enzyme, Type 3 Deiodinase, Is Essential for Coordination of Keratinocyte Growth and Differentiation. Thyroid. 2020; 30(7): 1066-1078.
43. Hogan M C, Griffin M D, Rossetti S, Torres V E, Ward C J, Harris P C. PKHDL1, a homolog of the autosomal recessive polycystic kidney disease gene, encodes a receptor with inducible T lymphocyte expression. Hum Mol Genet. 2003; 12(6):685-698.
44. Wu X, Ivanchenko M V, Al Jandal H, Cicconet M, Indzhykulian A A, Corey D P. PKHD1L1 is a coat protein of hair-cell stereocilia and is required for normal hearing. Nat Commun. 2019; 10(1):3801.
45. Ferrara A M, Liao X H, Gil-Ibanez P, Marcinkowski T, Bernal J, Weiss R E, Dumitrescu A M, Refetoff S. Changes in Thyroid Status During Perinatal Development of MCT8-Deficient Male Mice. Endocrinology 2013; 154(7):2533-2541.
46. Willnow T E, Christ A. Endocytic receptor LRP2/megalin-of holoprosencephaly and renal Fanconi syndrome. Pflugers Arch. 2017; 469(7-8):907-916.
47. Fass D, Blacklow S, Kim P S, Berger J M. Molecular basis of familial hypercholesterolaemia from structure of LDL receptor module. Nature. 1997; 388(6643):691-693.
48. Lisi S, Segnani C, Mattii L, Botta R, Marcocci C, Dolfi A, McCluskey R T, Pinchera A, Bernardini N, Marinò M. Thyroid dysfunction in megalin deficient mice. Mol Cell Endocrinol. 2005; 236(1-2):43-47.
49. Lisi S, Pinchera A, McCluskey R T, Willnow T E, Refetoff S, Marcocci C, Vitti P, Menconi F, Grasso L, Luchetti F, Collins A B, Marino M. Preferential megalin-mediated transcytosis of low-hormonogenic thyroglobulin: A control mechanism for thyroid hormone release. Proceedings of the National Academy of Sciences. 2003; 100(25): 14858-14863.
50. Kantarci S, Al-Gazali L, Hill R S, Donnai D, Black GCM, Bieth E, Chassaing N, Lacombe D, Devriendt K, Teebi A, Loscertales M, Robson C, Liu T, MacLaughlin D T, Noonan K M, Russell M K, Walsh C A, Donahoe P K, Pober B R. Mutations in LRP2, which encodes the multiligand receptor megalin, cause Donnai-Barrow and facio-oculo-acoustico-renal syndromes. Nature Genetics. 2007; 39(8):957-959.
51. Coscia F, Taler-Verčič A, Chang V T, Sinn L, O'Reilly F J, Izoré T, Renko M, Berger I, Rappsilber J, Turk D, Lowe J. The structure of human thyroglobulin. Nature. 2020; 578(7796): 627-630.
52. Xie J, Pannain S, Pohlenz J, Weiss R E, Moltz K, Morlot M, Asteria C, Persani L, Beck-Peccoz P, Parma J, Vassart G, Refetoff S. Resistance to thyrtropin (TSH) in three families is not associated with mutations in the TSH receptor or TSH. J Clin Endocrinol Metab. 1997; 82(12):3933-3940.
53. SGCappola P, RMA A. meta-analysis of thyroid-related traits reveals novel loci and gender-specific differences in the regulation of thyroid function. PLoS Genetics 20139 e1003266.
54. Tenenbaum-Rakover Y, Almashanu S, Hess O, Admoni O, Hag-Dahood Mahameed A, Schwartz N, Allon-Shalev S, Bercovich D, Refetoff S. Long-term outcome of loss-of-function mutations in thyrotropin receptor gene. Thyroid. 2015; 25(3):292-299.
55. Lair S, Crawshaw G J, Mehren K G, Perrone M A. Evaluation of a human immunometric assay for the determination of thyroid-stimulating hormone in nonhuman primates. J Zoo Wildl Med. 2000; 31(2):267-268.
56. Tarabichi M, Salcedo A, Deshwar A G, Ni Leathlobhair M, Wintersinger J, Wedge D C, Van Loo P, Morris Q D, Boutros P C. A practical guide to cancer subclonal reconstruction from DNA sequencing. Nat Methods. 2021; 18(2):144-155.
57. van der Vaart J, Bosmans L, Sijbesma S F, Knoops K, van de Wetering W J, Otten H G, Begthel H, Borel Rinkes IHM, Korving J, Lentjes E, Lopez-Iglesias C, Peters P J, van Santen H M, Vriens M R, Clevers H. Adult mouse and human organoids derived from thyroid follicular cells and modeling of Graves' hyperthyroidism. Proc Natl Acad Sci USA. 2021; 118(51).
58. Okita K, Matsumura Y, Sato Y, Okada A, Morizane A, Okamoto S, Hong H, Nakagawa M, Tanabe K, Tezuka K, Shibata T, Kunisada T, Takahashi M, Takahashi J, Saji H, Yamanaka S. A more efficient method to generate integration-free human iPS cells. Nat Methods. 2011; 8(5):409-412.
59. Meerbrey K L, Hu G, Kessler J D, Roarty K, Li M Z, Fang J E, Herschkowitz J I, Burrows A E, Ciccia A, Sun T, Schmitt E M, Bernardi R J, Fu X, Bland C S, Cooper T A, Schiff R, Rosen J M, Westbrook T F, Elledge S J. The pINDUCER lentiviral toolkit for inducible RNA interference in vitro and in vivo. Proc Natl Acad Sci USA. 2011; 108(9):3665-3670.
60. Allan W, Herndon C N, Dudley F C. Some examples of the inheritance of mental deficiency: apparently sex-linked idiocy and microcephaly. Am J Ment Defic. 1944; 48:325-334.
61. Schwartz C E, May M M, Carpenter N J, Rogers R C, Martin J, Bialer M G, Ward J, Sanabria J, Marsa S, Lewis J A, Echeverri R, Lubs H A, Voeller K, Simensen R J, Stevenson R E. Allan-Herndon-Dudley Syndrome and the Monocarboxylate Transporter 8 (MCT8) Gene. Am J Hum Genet. 2005; 77(1):41-53.
62. Dumitrescu A M, Liao X H, Weiss R E, Millen K, Refetoff S. Tissue specific thyroid hormone deprivation and excess in Mct8 deficient mice. Endocrinology 2006(147):4036-4043.
63. Trajkovic M, Visser T J, Mittag J, Horn S, Lukas J, Darras V M, Raivich G, Bauer K, Heuer H. Abnormal thyroid hormone metabolism in mice lacking the monocarboxylate transporter 8. J Clin Invest. 2007; 117(3): 627-635.
64. Roberts L M, Woodford K, Zhou M, Black D S, Haggerty J E, Tate E H, Grindstaff K K, Mengesha W, Raman C, Zerangue N. Expression of the thyroid hormone transporters MCT8 (SLC16A2) and OATP14 (SLCO1C1) at the blood-brain barrier. Endocrinology. 2008; 149(12): 6251-6261.
65. Mayerl S, Muller J, Bauer R, Richert S, Kassmann C M, Darras V M, Buder K, Boelen A, Visser T J, Heuer H. Transporters MCT8 and OATP1C1 maintain murine brain thyroid hormone homeostasis. J Clin Invest. 2014; 124(5): 1987-1999.
66. Vatine G D, Al-Ahmad A, Barriga B K, Svendsen S, Salim A, Garcia L, Garcia V J, Ho R, Yucer N, Qian T, Lim R G, Wu J, Thompson L M, Spivia W R, Chen Z, Van Eyk J, Palecek S P, Refetoff S, Shusta E V, Svendsen C N. Modeling Psychomotor Retardation using iPSCs from MCT8-Deficient Patients Indicates a Prominent Role for the Blood-Brain Barrier. Cell Stem Cell. 2017; 20(6):831-843 e835.
67. Vatine G D, Barrile R, Workman M J, Sances S, Barriga B K, Rahnama M, Barthakur S, Kasendra M, Lucchesi C, Kerns J, Wen N, Spivia W R, Chen Z, Van Eyk J, Svendsen C N. Human iPSC-Derived Blood-Brain Barrier Chips Enable Disease Modeling and Personalized Medicine Applications. Cell Stem Cell. 2019; 24(6):995-1005 e1006.
68. van Trotsenburg P, Stoupa A, Leger J, Rohrer T, Peters C, Fugazzola L, Cassio A, Heinrichs C, Beauloye V, Pohlenz J, Rodien P, Coutant R, Szinnai G, Murray P, Bartes B, Luton D, Salerno M, de Sanctis L, Vigone M, Krude H, Persani L, Polak M. Congenital Hypothyroidism: A 2020-2021 Consensus Guidelines Update-An ENDO-European Reference Network Initiative Endorsed by the European Society for Pediatric Endocrinology and the European Society for Endocrinology. Thyroid. 2021; 31(3):387-419.
69. Di Cosmo C, Liao X H, Dumitrescu A M, Weiss R E, Refetoff S. A thyroid hormone analogue with reduced dependence on the monocarboxylate transporter 8 (MCT8) for tissue transport. Endocrinology. 2009; 150(9):4450-4458.
70. Kersseboom S, Horn S, Visser W E, Chen J, Friesema E C, Vaurs-Barriere C, Peeters R P, Heuer H, Visser T J. In vitro and mouse studies supporting therapeutic utility of triiodothyroacetic acid in MCT8 deficiency. Mol Endocrinol. 2014; 28(12):1961-1970.
71. Groeneweg S, Peeters R P, Moran C, Stoupa A, Auriol F, Tonduti D, Dica A, Paone L, Rozenkova K, Malikova J, van der Walt A, de Coo IFM, McGowan A, Lyons G, Aarsen F K, Barca D, van Beynum I M, van der Knoop M M, Jansen J, Manshande M, Lunsing R J, Nowak S, den Uil C A, Zillikens M C, Visser F E, Vrijmoeth P, de Wit M C Y, Wolf N I, Zandstra A, Ambegaonkar G, Singh Y, de Rijke Y B, Medici M, Bertini E S, Depoorter S, Lebl J, Cappa M, De Meirleir L, Krude H, Craiu D, Zibordi F, Oliver Petit I, Polak M, Chatterjee K, Visser T J, Visser W E. Effectiveness and safety of the tri-iodothyronine analogue Triac in children and adults with MCT8 deficiency: an international, single-arm, open-label, phase 2 trial. Lancet Diabetes Endocrinol. 2019; 7(9):695-706.
72. Mendell J R, Al-Zaidy S A, Lehman K J, McColly M, Lowes L P, Alfano L N, Reash N F, Iammarino M A, Church K R, Kleyn A, Meriggioli M N, Shell R. Five-Year Extension Results of the Phase 1 START Trial of Onasemnogene Abeparvovec in Spinal Muscular Atrophy. JAMA Neurol. 2021; 78(7):834-841.
73. Di Cosmo C, Liao X H, Ye H, Ferrara A M, Weiss R E, Refetoff S, Dumitrescu A M. Mct8-deficient mice have increased energy expenditure and reduced fat mass that is abrogated by normalization of serum T3 levels. Endocrinology. 2013; 154(12):4885-4895.
74. Leung E K, Yi X, Refetoff S, Yeo K T. Diiodothyropropionic acid (DITPA) cross-reacts with thyroid function assays on different immunoassay platforms. Clin Chim Acta. 2016; 453:203-204.
75. Chan S L, Refetoff S, Babic N, Jin M, Garg U, Yeo K J. Triiodothyroacetic Acid Cross-Reacts With Measurement of Triiodothyronine (T₃) on Various Immunoassay Platforms. Am J Clin Pathol. 2021; 157(2):156-158.
76. Juge-Aubry C E, Morin O, Pernin A T, Liang H, Philippe J, Burger A G. Long-lasting effects of Triac and thyroxine on the control of thyrotropin and hepatic deiodinase type I. Eur J Endocrinol. 1995; 132(6):751-758.
77. Calvo R, Obregon M J, Ruiz de Ona C, Escobar del Rey F, Morreale de Escobar G. Congenital hypothyroidism, as studied in rats. Crucial role of maternal thyroxine but not of 3,5,3′-triiodothyronine in the protection of the fetal brain. J Clin Invest. 1990; 86(3):889-899.

It is to be understood that the subject matter herein is not limited to the specific embodiments described above but encompasses any and all embodiments within the scope of the generic language of the following claims enabled by the embodiments described herein, or otherwise described above in terms sufficient to enable one of ordinary skill in the art to make and use the claimed subject matter.

We claim:

1. A method of treating Allan-Herndon-Dudley syndrome, the method comprising
determining that a subject is in need of treatment for Allan-Herndon-Dudley syndrome;
and administering gene therapy to introduce normal human MCT8 into the subject's cells to increase T3 in the subject's brain and induce a T3-mediated response.

2. The method of claim 1, wherein the normal human MCT8 is introduced using a viral vector AAV9.

3. The method of claim 1, further comprising administration of DITPA to the subject.

4. The method of claim 2, further comprising administration of DITPA to the subject.

5. The method of claim 3, wherein the administration of DITPA to the subject begins at one dose for two weeks and continues for at least two weeks at a higher dose.

6. The method of claim 4, wherein the administration of DITPA to the subject begins at one dose for two weeks and continues for at least two weeks at a higher dose.

7. The method of claim 5, wherein each of the one dose and the higher dose administered to the subject is determined based on T3 serum levels of the subject.

8. The method of claim 3, further comprising administration of TRIAC to the subject.

9. The method of claim 4, further comprising administration of TRIAC to the subject.

10. The method of claim 5, further comprising administration of TRIAC to the subject.

11. The method of claim 6, further comprising administration of TRIAC to the subject.
